SEQUENCE LISTING (8 sequences)

SEQ ID NO: 1 - DNA, 1527 bp, Homo sapiens
ggggatgtggcccgtggcctagctcgtcaagttgccgtggcgcggagaactctgcaaaacaagaggctgaggattgcgttagagataaaccagttcacgccggagccccgtgagggaagcgtctccgttgggtccggccgctctgcgggactctgaggaaaagctcgcaccaggtggacgcggatctgtcaacatgggtaaaggagaccccaacaagccgcggggcaaaatgtcctcgtacgccttcttcgtgcagacctgccgggaagagcacaagaagaaacacccggactcttccgtcaatttcgcggaattctccaagaagtgttcggagagatggaagaccatgtctgcaaaggagaagtcgaagtttgaagatatggcaaaaagtgacaaagctcgctatgacagggagatgaaaaattacgttcctcccaaaggtgataagaaggggaagaaaaaggaccccaatgctcctaaaaggccaccatctgccttcttcctgttttgctctgaacatcgcccaaagatcaaaagtgaacaccctggcctatccattggggatactgcaaagaaattgggtgaaatgtggtctgagcagtcagccaaagataaacaaccatatgaacagaaagcagctaagctaaaggagaaatatgaaaaggatattgctgcatatcgtgccaagggcaaaagtgaagcaggaaagaagggccctggcaggccaacaggctcaaagaagaagaacgaaccagaagatgaggaggaggaggaggaagaagaagatgaagatgaggaggaagaggatgaagatgaagaataaatggctatcctttaatgatgcgtgtggaatgtgtgtgtgtgctcaggcaattattttgctaagaatgtgaattcaagtgcagctcaatactagcttcagtataaaaactgtacagatttttgtatagctgataagattctctgtagagaaaatacttttaaaaaatgcaggttgtagctttttgatgggctactcatacagttagattttacagcttctgatgttgaatgttcctaaatatttaatggtttttttaatttcttgtgtatggtagcacagcaaacttgtaggaattagtatcaatagtaaattttgggttttttaggatgttgcatttcgtttttttaaaaaaaattttgtaataaaattatgtatattatttctattgtctttgtcttaatatgctaagttaattttcactttaaaaaagccatttgaagaccagagctatgttgatttttttcggtatttctgcctagtagttcttagacacagttgacctagtaaaatgtttgagaattaaaaccaaacatgctcatatttgcaaaatgttctttaaaagttacatgttgaactcagtgaactttataagaatttatgcagttttacagaacgttaagttttgtacttgacgtttctgtttattagctaaattgttcctcaggtgtgtgtatatatatatacatatatatatatatatatat

SEQ ID NO: 2 - DNA, 1467 bp, Homo sapiens
aaaccagttcacgccggagccccgtgagggaagcgtctccgttgggtccggccgctctgcgggactctgaggaaaagctcgcaccaggcaagaataccctccaataccctcgggtggacgcggatctgtcaacatgggtaaaggagaccccaacaagccgcggggcaaaatgtcctcgtacgccttcttcgtgcagacctgccgggaagagcacaagaagaaacacccggactcttccgtcaatttcgcggaattctccaagaagtgttcggagagatggaagaccatgtctgcaaaggagaagtcgaagtttgaagatatggcaaaaagtgacaaagctcgctatgacagggagatgaaaaattacgttcctcccaaaggtgataagaaggggaagaaaaaggaccccaatgctcctaaaaggccaccatctgccttcttcctgttttgctctgaacatcgcccaaagatcaaaagtgaacaccctggcctatccattggggatactgcaaagaaattgggtgaaatgtggtctgagcagtcagccaaagataaacaaccatatgaacagaaagcagctaagctaaaggagaaatatgaaaaggatattgctgcatatcgtgccaagggcaaaagtgaagcaggaaagaagggccctggcaggccaacaggctcaaagaagaagaacgaaccagaagatgaggaggaggaggaggaagaagaagatgaagatgaggaggaagaggatgaagatgaagaataaatggctatcctttaatgatgcgtgtggaatgtgtgtgtgtgctcaggcaattattttgctaagaatgtgaattcaagtgcagctcaatactagcttcagtataaaaactgtacagatttttgtatagctgataagattctctgtagagaaaatacttttaaaaaatgcaggttgtagctttttgatgggctactcatacagttagattttacagcttctgatgttgaatgttcctaaatatttaatggtttttttaatttcttgtgtatggtagcacagcaaacttgtaggaattagtatcaatagtaaattttgggttttttaggatgttgcatttcgtttttttaaaaaaaattttgtaataaaattatgtatattatttctattgtctttgtcttaatatgctaagttaattttcactttaaaaaagccatttgaagaccagagctatgttgatttttttcggtatttctgcctagtagttcttagacacagttgacctagtaaaatgtttgagaattaaaaccaaacatgctcatatttgcaaaatgttctttaaaagttacatgttgaactcagtgaactttataagaatttatgcagttttacagaacgttaagttttgtacttgacgtttctgtttattagctaaattgttcctcaggtgtgtgtatatatatatacatatatatatatatatatat

SEQ ID NO: 3 - DNA, 1454 bp, Homo sapiens
ggatttgggcgggaagcggagccccgccagcgcccgccctggcagctgcgggctccgcgccgaccctccggcttcccctctcccccctcggccccgtcaggtggacgcggatctgtcaacatgggtaaaggagaccccaacaagccgcggggcaaaatgtcctcgtacgccttcttcgtgcagacctgccgggaagagcacaagaagaaacacccggactcttccgtcaatttcgcggaattctccaagaagtgttcggagagatggaagaccatgtctgcaaaggagaagtcgaagtttgaagatatggcaaaaagtgacaaagctcgctatgacagggagatgaaaaattacgttcctcccaaaggtgataagaaggggaagaaaaaggaccccaatgctcctaaaaggccaccatctgccttcttcctgttttgctctgaacatcgcccaaagatcaaaagtgaacaccctggcctatccattggggatactgcaaagaaattgggtgaaatgtggtctgagcagtcagccaaagataaacaaccatatgaacagaaagcagctaagctaaaggagaaatatgaaaaggatattgctgcatatcgtgccaagggcaaaagtgaagcaggaaagaagggccctggcaggccaacaggctcaaagaagaagaacgaaccagaagatgaggaggaggaggaggaagaagaagatgaagatgaggaggaagaggatgaagatgaagaataaatggctatcctttaatgatgcgtgtggaatgtgtgtgtgtgctcaggcaattattttgctaagaatgtgaattcaagtgcagctcaatactagcttcagtataaaaactgtacagatttttgtatagctgataagattctctgtagagaaaatacttttaaaaaatgcaggttgtagctttttgatgggctactcatacagttagattttacagcttctgatgttgaatgttcctaaatatttaatggtttttttaatttcttgtgtatggtagcacagcaaacttgtaggaattagtatcaatagtaaattttgggttttttaggatgttgcatttcgtttttttaaaaaaaattttgtaataaaattatgtatattatttctattgtctttgtcttaatatgctaagttaattttcactttaaaaaagccatttgaagaccagagctatgttgatttttttcggtatttctgcctagtagttcttagacacagttgacctagtaaaatgtttgagaattaaaaccaaacatgctcatatttgcaaaatgttctttaaaagttacatgttgaactcagtgaactttataagaatttatgcagttttacagaacgttaagttttgtacttgacgtttctgtttattagctaaattgttcctcaggtgtgtgtatatatatatacatatatatatatatatatat

SEQ ID NO: 4 - DNA, 97 bp, Homo sapiens
tgtgatcactgtctccagcctgctgaagctcagagggctctgattcagaaagatcatcggatccgtctgagcttggctggtcggaagtctcatcatc

SEQ ID NO: 5 - DNA, 78 bp, Homo sapiens
atacttgaggagaaattatccttggtgtgttcgctttatttatgatgaatcatacaaggacaatttctttttgagtat

SEQ ID NO: 6 - DNA, 78 bp, Homo sapiens
tacttaatgagaagttgcccgtgtttttttcgctttatttgtgacgaaacattcgcggtgcacttctttttcagtatc

SEQ ID NO: 7 - DNA, 73 bp, Homo sapiens
ggccggctggggttcctggggatgggatttgcttcctgtcacaaatcacattgccagggatttccaaccgacc

SEQ ID NO: 8 - DNA, 58 bp, Artificial Sequence
  shRNA-HMGB2
ccgggctcaacattagcttcagtatctcgagatactgaagctaatgttgagctttttg

(19) United States
(12) Patent Application Publication
(10) Pub. No.: US 2018/0320177 A1
(43) Pub. Date: Nov. 8, 2018

(54) COMPOSITIONS AND METHODS FOR THE TREATMENT OF LIVER FIBROSIS

(71) Applicant: UNIVERSITY OF CONNECTICUT, FARMINGTON, CT (US)

(72) Inventors: LI WANG, ELLINGTON, CT (US); DENNIS L. WRIGHT, UNIONVILLE, CT (US); DIANE J. BURGESS, STORRS, CT (US)

(21) Appl. No.: 15/773,647

(22) PCT Filed: Nov. 2, 2016
(86) PCT No.: PCT/US16/60028

§ 371 Date: May 4, 2018

Related U.S. Application Data
(60) Provisional application No. 62/251,380, filed on Nov. 5, 2015.

(51) Int. Cl. C12N 15/113; A61K 31/5025; A61K 31/7105; A61K 9/127; A61P 1/16

(52) U.S. Cl. CPC C12N 15/113; A61K 31/5025; A61K 31/7105; C12N 2310/122; A61P 1/16; C12N 2310/14; C12N 2310/141; A61K 9/1272

(57) ABSTRACT

Described herein are methods of treating a subject in need of treatment for liver fibrosis by administering a therapeutically effective amount of an inhibitor of high mobility box 2 gene (HMGB2). In an aspect, a composition comprises an inflachromene analog and a micro RNA or an interfering RNA that inhibits the expression of HMGB2. Also described is a composition including an anionic or cationic liposome particle, containing Vitamin A, and an inhibitor of HMGB2.

### FIELD OF THE DISCLOSURE

The present disclosure is related to compositions and methods for the treatment and prevention of liver fibrosis and other liver disorders.

### BACKGROUND

Liver cirrhosis, or fibrosis, the ultimate pathological feature of all forms of chronic hepatic damage, is responsible for much morbidity and mortality worldwide, causing around 1.5 million deaths per year. Such damage can be the result of viral activity (e.g., chronic hepatitis types B or C) or other liver infections (e.g., parasites, bacteria); exposure to chemicals (e.g., pharmaceuticals, recreational drugs, excessive alcohol, exposure to pollutants); autoimmune processes (e.g., autoimmune hepatitis); metabolic disorders (e.g., lipid, glycogen, cholesterol or metal storage disorders); or cancer growth (primary or secondary liver cancer). Liver fibrosis is present in virtually all patients with chronic liver injury, regardless of the etiology. The principal cell type responsible for liver fibrosis is the hepatic stellate cell (HSC), a resident perisinusoidal cell that takes up vitamin A from circulation and stores it.

Early treatment is aimed at causes such as antiviral treatment or alcohol detoxification, however, if damage has progressed sufficiently there is no currently available treatment to reverse the fibrotic consequence of chronic liver insults. The majority of patients with chronic liver disease will ultimately progress over decades to advanced fibrosis or cirrhosis, or liver cancer. Much progress has been made in understanding the molecular basis of liver fibrosis. However, identifying new regulators and treatment methods is crucial to the development of treatments for liver fibrosis and cirrhosis.

### BRIEF SUMMARY

In one aspect, a method of treating a subject in need of treatment for liver fibrosis comprises administering a therapeutically effective amount of an inhibitor of high mobility box 2 gene (HMGB2).

In another aspect, a composition comprises an inflachromene analog of Formula I and a micro RNA or an interfering RNA that inhibits the expression of HMGB2,

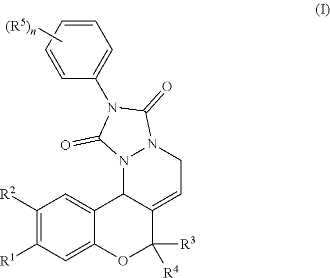

wherein

R¹ and R² are each independently hydrogen, —OH, C₁-C₆ alkyl, C₁-C₆ alkoxy, C₂-C₆ alkenyl, C₂-C₆ alkynyl, C₂-C₆ alkanoyl, C₂-C₆ alkoxycarbonyl, mono- or di-C₁-C₆ alkylcarboxamide, mono- or di-C₁-C₆ alkylamino, aryl, (aryl)C₁-C₆ alkyl, substituted or unsubstituted (aryl)carbonyl, C₃-C₈ cycloalkyl, halogen, C₁-C₆ haloalkyl, C₁-C₆ haloalkoxy, heteroaryl, (heteroaryl)C₁-C₆ alkyl, heterocycloalkyl, —COOH, —CN, —NH₂, —NO₂, (C₁-C₆ alkyl)amido, (C₂-C₆ alkenyl)amido, or (C₂-C₆ alkynyl)amido;

R³ and R⁴ are each independently hydrogen, —OH, C₁-C₆ alkyl, C₁-C₆ alkoxy, C₂-C₆ alkenyl, C₂-C₆ alkynyl, C₂-C₆ alkanoyl, C₂-C₆ alkoxycarbonyl, mono- or di-C₁-C₆ alkylcarboxamide, mono- or di-C₁-C₆ alkylamino, aryl, (aryl)C₁-C₆ alkyl, substituted or unsubstituted (aryl)carbonyl, C₃-C₈ cycloalkyl, halogen, C₁-C₆ haloalkyl, C₁-C₆ haloalkoxy, heteroaryl, (heteroaryl)C₁-C₆ alkyl, heterocycloalkyl, —COOH, —CN, —NH₂, —NO₂, (C₁-C₆ alkyl)amido, (C₂-C₆ alkenyl)amido, (C₂-C₆ alkynyl)amido, or R³ and R⁴ together can form a substituted or unsubstituted C₃-C₆ cycloalkyl, or substituted or unsubstituted C₃-C₆ heterocycloalkyl;

each instance of R⁵ is hydrogen, —OH, C₁-C₆ alkyl, C₁-C₆ alkoxy, C₂-C₆ alkenyl, C₂-C₆ alkynyl, C₂-C₆ alkanoyl, C₂-C₆ alkoxycarbonyl, mono- or di-C₁-C₆ alkylcarboxamide, mono- or di-C₁-C₆ alkylamino, aryl, (aryl)C₁-C₆ alkyl, substituted or unsubstituted (aryl)carbonyl, C₃-C₈ cycloalkyl, halogen, C₁-C₆ haloalkyl, C₁-C₆ haloalkoxy, heteroaryl, (heteroaryl)C₁-C₆ alkyl, heterocycloalkyl, —COOH, —CN, —NH₂, —NO₂, (C₁-C₆ alkyl)amido, (C₂-C₆ alkenyl)amido, or (C₂-C₆ alkynyl)amido; n is 0, 1, 2, or 3; or a pharmaceutically acceptable salt thereof; with the proviso that when R² and R⁵ are both hydrogen then R³, and R⁴ are not —OH, methyl, and methyl, respectively.

In yet another aspect, a composition comprises an anionic or catioinic liposome particle comprising Vitamin A, and an inhibitor of HMGB2.

### BRIEF DESCRIPTION OF THE DRAWINGS

FIG. 1 shows high mobility box 2 (HMGB2) expression is highly induced in human fibrotic and cirrhotic livers as determined by qPCR analysis. It is also induced in mouse models of liver fibrosis.

FIGS. 2A-C show that the expression of HMGB2 is highly induced in mouse fibrotic livers. FIG. 2A shows Genome Browser visualization tracks from RNA-seq reads depict altered hepatic HMGB2 expression from embryonic day 14 (E14) to postnatal day 60 (P60). FIG. 2B shows qPCR of HMGB2 mRNA in mouse liver from E12 to P60. FIG. 2C shows Western blot of HMGB2 protein in mouse fibrotic liver induced by carbon tetrachloride (CCl4).

FIGS. 3A and B show a Western blot of HMGB2 protein (FIG. 3A) and qPCR of HMGB2 mRNA (FIG. 3B) in human NASH and alcohol cirrhotic (cir) livers. * p<0.01 vs. normal (Nor).

FIGS. 4A-D show that overexpression of HMGB2 enhances CCl₄-induced inflammation (FIG. 4A), increases mTOR pathway and decrease p53 pathway (FIG. 4B). Knockdown HMGB2 by shRNA (FIG. 4C) or ICM (FIG. 4D) decrease inflammatory response.

FIGS. 5A-D show that overexpression of HMGB2 initiates liver fibrosis and enhances CCl₄-induced liver fibrosis, whereas HMGB2-deficiency (HMGB2−/−) prevents liver fibrosis and serum ALT levels (FIG. 5A). ICM decreases HMGB2 protein, liver fibrosis and serum ALT (FIG. 5B). In LX2 cells, ICM decreases Smad signaling and reduces cytoplasm HMGB2 protein (FIG. 5C). ICM decreases HMGB2 acetylation by CBP/CCl₄ and phosphorylation by CCl₄ (FIG. 5D).

FIGS. 6A-F show an established primary stellate cell culture (FIG. 6A). HMGB2−/− primary stellate cells have delayed differentiation and alpha-SMA activation (FIG. 6B). The activation of pAKT and pERK1/2 is diminished in HMGB2−/− stellate cells cultured in vitro (FIG. 6C). HMGB2−/− primary stellate cells show diminished response to PDGF-induced pAKT and pERK1/2 activation (FIG. 6D). ICM reduces pAKR and pERk1/2 activation and HMGB2 protein (FIG. 6E). CCl₄ significantly induces HMGB2 protein which is blocked by ICM (FIG. 6F). When stopping CCl4 feeding (4w, stop), liver fibrosis is reversed and HMGB2 protein, but not HMGB1 protein, is reduced to basal level.

FIGS. 7A-F show that HMGB2 protein is highly induced in liver tumors and liver progenitor cell TIC cells (FIG. 7A). Generating HMGB2 stable knockdown TIC cells (FIG. 7B). shHMGB2 cells show decreased cell migration and invasion, which are reversed by HMGB2 overexpression (FIG. 7C). ICM markedly blocks TIC cell migration and invasion (Figure D). shHMGB2 or ICM treated cells show decreased MMP and Rho signaling (FIG. 7E). shHMGB2 cells show decreased spheroid size (FIG. 7F).

FIGS. 8A-C show that mouse serum HMGB2 protein levels are increased in 10 wks CCl₄ treated mice (more severe stage of liver fibrosis) (FIG. 8A). In human serum from alcoholic liver cirrhosis (ALD), HepC and NASH cirrhosis patients, HMGB2 protein levels are markedly increased (FIG. 8B-FIG. 8C).

FIGS. 9A-D show that high HMGB2 expression is correlated with human NASH cirrhosis (FIG. 9A) and advanced fibrosis (FIG. 9B). HMGB2 expression is upregulated in human metastasis HCC (FIG. 9C) and its high expression is correlated with poor patient survival (FIG. 9D).

FIGS. 10A-E show the establishment of CGR8 cell culture and differentiation (FIG. 10A-C). FIG. 10B shows qPCR of ES markers and three-germ layer genes mRNA in undifferentiated CGR8 ES cells. FIG. 10C shows qPCR of ES markers and three-germ layer genes mRNA during ES cell differentiation. FIG. 10D shows Western Blot of ES markers and HMGB2 during ES cell differentiation. FIG. 10E shows qPCR of miR-127, Shp and Eid1 mRNA during ES cell differentiation.

FIG. 11 shows inhibition of HMGB2 protein by miRNAs in both human and mouse cells.

FIGS. 12A-F show that miR-127 targets the HMGB2 3′UTR show that miR-127 targets the HMGB2 3′UTR and decreases HMGB2 protein in HCC cells (FIGS. 12C-F). FIG. 12 A is a sequence alignment of miR-127 and the 3′-UTR of HMGB2 in human, mouse and rat. FIG. 12B is a Luciferase reporter assay. In FIG. 12C, Western blotting was performed to determine endogenous HMGB2 protein in Hela cells that were transfected with pTarget, pTarget-miR-433, or pTarget-miR-127. (3-Actin served as an internal control. In FIG. 12D, Left, western blot was performed to determine HMGB2 and CREB protein level in MHCC97H cells transfected with miR-127 for 24 hrs. Right, qPCR analysis of miR-127 level in MHCC97 cells transfected with miR-127 for 0, 24, 48 hrs. In FIG. 12E, Left, western blot was performed to determine HMGB2 and CREB protein level in HepG2 cells transfected with miR-127 for 0, 24, 48 hrs. Right, qPCR analysis of miR-127 level in HepG2 cells transfected with miR-127 for 0, 24, 48 hrs. In FIG. 12F, Left, western blot was performed to determine HMGB2 protein level in HepG2 cells transfected with or without miR-127 antagomir (anti-miR-127) for 48 hrs. Right, qPCR analysis of miR-127 level in HepG2 cells transfected with anti-miR-127 for 48 hrs.

FIGS. 13A-E show HMGB2 protein is E2F1/SHP/EID1 network. FIG. 13 A is a Western blot of HMGB2 protein in wild-type or Shp-deficiency mouse liver. FIG. 13B is a Western blot (Left) and qPCR (Right) of HMGB2 in wild-type or Shp-deficiency mouse liver treated with or without EtOH. FIG. 13C is a Luciferase reporter assay. Left, HEK293T cells were transfected with HMGB2 promoter reporter with 0, 50 or 100 ng E2F1 expression vector. Right, HEK293T cells were transfected with HMGB2 promoter reporter with E2F1, SHP and EID expression plasmid alone or in combination. Luciferase activity was normalized to Renilla activity. FIG. 13D is a qPCR of HMGB2 mRNA in HEK293T cells that transfected with E2F1, SHP and EID1 expression vectors alone or in combination. FIG. 13E is a Western blot of HMGB2 in wild-type or E2f1-deficiency mouse liver fed with nor, Chow or DDC diet.

FIG. 14 HMGB2 modulates ES pluripotency via stabilizing OCT4 and SOX2. FIG. 14A is Left Western blot of HMGB2 and SOX2 protein in HEK293T cells transfected with 0.5 μg HA-SOX2 expression plasmid and 0, 1 or 2 μg Flag-HMGB2 expression plasmid. Right, Western blot of HMGB2 and OCT4 protein in HEK293T cells transfected with 0.5 μg HA-OCT4 expression plasmid and 0, 1 or 2 μg Flag-HMGB2 expression plasmid. FIG. 14B shows Endogenous co-immunoprecipitation. CGR8 cells were lysed, co-immunoprecipitated with OCT4 antibody, followed by Western blot with HMGB2 or OCT4 antibodies. FIG. 14C shows a Luciferase reporter assay. FIG. 14D shows Microphage of EB formation from CGR8 cells infected with AAV8-Null or AAV8-shHMGB2. FIG. 14E shows Microphage of TIC cells (Upper) or CGR8 cells (Bottom) treated with 5 □M HMGB2 inhibitor Inflachromene (ICM) or DMSO.

FIGS. 15A-C show Mir-127 modulates ES cell pluripotency and TIC cell via targeting HMBG2. FIG. 15A shows qPCR of ES markers and three-germ layer genes mRNA in undifferentiated CGR8 ES cells and 4d EBs which was infected with miR-127 or GFP lentivirus. FIG. 15B shows Left, qPCR analysis of miR-127 level in TIC cells which were infected with miR-127 lentivirus. Right, western blot of HMGB2, RhoA, CDC42, Rac1/2/3 in TIC cells which were infected with miR-127 or GFP lentivirus. FIG. 15C shows a spheroid assay of TIC cells infected with miR-127, GFP or miR-127 combined with HMGB2 lentivirus.

FIGS. 16A-D show diminishing liver fibrosis by HMGB2 inhibitor inflachromene (ICM). FIG. 16A is a schematic of CCl₄ and ICM (in PEG: Polyethylene Glycol, 10 mg/kg bw, i.p. daily for 4 wks) injection. FIG. 16B shows gross morphology of mouse liver treated with CCl₄ or CCl₄+ICM (top) and WB of HMGB2 and HMGB1 protein (CCl₄ group: n=3; CCl₄+ICM group: n=5) (bottom). FIG. 16C shows plasma ALT levels. FIG. 16D shows Masson Trichrome staining of liver sections (left) and quantification of liver fibrosis (right).

FIGS. 17A-C show diminishing liver fibrosis by shHMGB2. FIG. 17A is a schematic showing liposome injection (20 μg/mouse, twice/wk). FIGS. 17B shows WB of HMGB2 and HMGB1 protein (lipo-CCl4 group: n=5; lipo-shHMGB2 group: n=3). Lipo: liposome. FIG. 17C shows Masson Trichrome staining of liver sections.

FIG. 18 shows the action of ICM may be tissue or cell type specific. Left: WB of HMGB2 protein in LX-2 and Huh7 cells treated with ICM. Fresh media every day for 3 days. Right: Masson Trichrome staining of liver sections.

FIG. 19 is a schematic diagram for the generation of ICM analogs.

FIG. 20 shows a schematic of stellate cell-targeted liposomes (containing the drug: a small molecule inhibitor) complexed with siRNA or mRNA via a positive charged species (e.g., Ca²⁺).

The above-described and other features will be appreciated and understood by those skilled in the art from the following detailed description, drawings, and appended claims.

### DETAILED DESCRIPTION

The present inventors have unexpectedly found that the high mobility box 2 gene (HMGB2) is highly induced in various liver fibrosis models in humans and mice. Thus, HMGB2 is a novel target for the treatment and prevention of liver fibrosis and other liver disorders. Described herein are methods, uses, formulations and compositions to selectively target HMGB2 for treatment and prevention of liver fibrosis.

High-mobility group box (HMGB) proteins are non-histone chromosomal proteins with diverse functions in the cell. HMGB1 and HMGB2 are highly conserved (with 80% amino acid identity) and have indistinguishable DNA binding properties without sequence specificity. The function of HMGB1 in inflammation, fibrosis and hepatocellular carcinoma (HCC) has been extensively characterized. Regarding HMGB2, one study reported that HMGB2 expression was increased in patients with HCC and was positively correlated with shorter overall survival times. The present inventors found that HMGB2 expression was highly induced in human fibrotic and cirrhotic liver (FIG. 1), suggesting an important and clinically relevant role. This is the first initiative to demonstrate that HMGB2 is a novel regulator of liver metabolic homeostasis using comprehensive experimental approaches and the newly developed HMGB2−/− mice.

The sequence of human HMGB2 has three variants: SEQ ID NO: 1, accession number NM_002129.3; Seq ID NO: 2, accession number NM_001130688; and SEQ ID NO: 3, accession number NM_001130689.

As used herein, an HMGB2 inhibitor is a molecule that inhibits the activity of HMGB2, either at the protein level or at the nucleic acid level. Thus, an HMGB2 inhibitor decreases the expression and/or activity of HMGB2. For example, HMGB2 inhibitors may target the HMGB2 mRNA, resulting in reduced expression of the HMGB2 protein. Exemplary inhibitors of HMGB2 mRNA include the microRNA miR-127 and the small hairpin RNA shRNA-HMGB2. Alternatively, HMGB2 inhibitors may directly interact with the HMGB2 protein, inhibiting its activity. Exemplary inhibitors of the HMGB2 protein include inflachromene and its analogs.

As used herein “decreases” means a reduction of at least 5% relative to a reference level. A decrease may be by 5%, 10%, 15%, 20%, 25% or 50%, or even by as much as 75%, 85%, 95% or more.

As used herein “increases” means a stimulation of at least 5% relative to a reference level. An increase may be by 5%, 10%, 15%, 20%, 25% or 50%, or even by as much as 75%, 85%, 95% or more.

In an aspect, a method of treating a patient in need of treatment for liver fibrosis comprises administering a therapeutically effective amount of an inhibitor of high mobility box 2 gene (HMGB2).

An “effective amount” is the amount of an agent required to ameliorate the symptoms of a disease or slow, stabilize, prevent, or reduce the severity of the pathology in a subject relative to an untreated subject. The effective amount of active agent(s) varies depending upon the manner of administration, the age, body weight, and general health of the subject. Ultimately, the attending physician or veterinarian will decide the appropriate amount and dosage regimen. Such amount is referred to as an “effective” amount.

Exemplary subjects for the methods described herein include mammalian subjects, particularly human subjects. Exemplary human subjects are subjects with chronic liver damage such as subjects diagnosed with chronic liver injury; subjects with cirrhosis; subjects with chronic viral disease such as subjects with hepatitis B, subjects with hepatitis C, or subjects with nonalcoholic steatohepatitis (NASH); subjects with a parasitic liver infection; subjects with a bacterial liver infection; subjects with primary or secondary liver cancer; subjects with metabolic disorders that cause liver injury; subjects with alcoholic liver disease; subjects with Wilson's disease; subjects with nonalcoholic fatty liver disease; subjects with autoimmune liver disease; subjects with cholestatic liver disease; and subjects with biliary obstruction.

In a specific aspect, the inhibitor of HMGB2 is an inhibitory nucleic acid, for example a nucleic acid that specifically binds at least a portion of the HMGB2 mRNA (e.g., an interfering RNA) or a non-coding RNA that regulates the expression of the HMGB2 mRNA (e.g., a micro RNA). Small interfering RNAs (siRNAs) and microRNAs (miRNAs) repress gene expression through nucleic acid base-pairing between the target mRNA and the small RNA guide bound to a member of the Argonaute family of proteins.

MicroRNAs are small non-coding RNAs that regulate the expression of protein-coding genes. MicroRNA expression becomes altered with the development and progression of ovarian cancer. As used herein interchangeably, a “miR gene product,” “microRNA,” “miR,” or “miRNA” refers to the unprocessed or processed RNA transcript from a miR gene.

The active 19-25 nucleotide miRNA molecule can be obtained from an miR precursor through natural processing routes (e.g., using intact cells or cell lysates) or by synthetic processing routes (e.g., using isolated processing enzymes, such as isolated Dicer, Argonaut, or RNAse III). The active 19-25 nucleotide miRNA molecule can also be produced directly by biological or chemical synthesis, without having to be processed from the miR precursor. When a microRNA is referred to herein by name, the name corresponds to both the precursor and mature forms, unless otherwise indicated.

In one aspect, the miRNA is miR-127, an miRNA whose expression has been linked to hepatocellular carcinoma. Additional miRNAs that can be used to inhibit HMGB2 include miR-539, miR-543, miR-23a

miR-127: accession number NR_029696.1
(SEQ ID NO: 4)
tgtgatcact gtctccagcc tgctgaagct cagagggctc
tgattcagaa agatcatcgg atccgtctga gcttggctgg
tcggaagtct catcatc
miR-539: accession number NR_030256.1
(SEQ ID NO: 5)
atacttgagg agaaattatc cttggtgtgt tcgctttatt
tatgatgaat catacaagga caatttcttt ttgagtat
miR-543: accession number NR_030619.1
(SEQ ID NO: 6)
tacttaatga gaagttgccc gtgttttttt cgctttattt
gtgacgaaac attcgcggtg cacttctttt tcagtatc
miR-23a: accession number NR_029495.1
(SEQ ID NO: 7)
ggccggctgg ggttcctggg gatgggattt gcttcctgtc
acaaatcaca ttgccaggga tttccaaccg acc

The term “inhibitory nucleic acid molecule” means a single stranded or double-stranded RNA or DNA, specifically RNA (e.g., an iRNA), such as triplex oligonucleotides, ribozymes, aptamers, small interfering RNA including siRNA (short interfering RNA) and shRNA (short hairpin RNA), antisense RNA, or a portion thereof, or an analog or mimetic thereof, that is capable of reducing or inhibiting the expression of a target gene or sequence. Inhibitory nucleic acids can act by, for example, mediating the degradation or inhibiting the translation of mRNAs which are complementary to the interfering RNA sequence. An inhibitory nucleic acid, when administered to a mammalian cell, results in a decrease (e.g., by 5%, 10%, 25%, 50%, 75%, or even 90-100%) in the expression (e.g., transcription or translation) of a target sequence. Typically, a nucleic acid inhibitor comprises or corresponds to at least a portion of a target nucleic acid molecule, or an ortholog thereof, or comprises at least a portion of the complementary strand of a target nucleic acid molecule. Inhibitory nucleic acids may have substantial or complete identity to the target gene or sequence, or may include a region of mismatch (i.e., a mismatch motif). The sequence of the inhibitory nucleic acid can correspond to the full-length target gene, or a subsequence thereof. In one aspect, the inhibitory nucleic acid molecules are chemically synthesized.

The specific sequence utilized in design of the inhibitory nucleic acids is a contiguous sequence of nucleotides contained within the expressed gene message of the target. Factors that govern a target site for the inhibitory nucleic acid sequence include the length of the nucleic acid, binding affinity, and accessibility of the target sequence. Sequences may be screened in vitro for potency of their inhibitory activity by measuring inhibition of target protein translation and target related phenotype, e.g., inhibition of cell proliferation in cells in culture. In general it is known that most regions of the RNA (5′ and 3′ untranslated regions, AUG initiation, coding, splice junctions and introns) can be targeted using antisense oligonucleotides. Programs and algorithms, known in the art, may be used to select appropriate target sequences. In addition, optimal sequences may be selected utilizing programs designed to predict the secondary structure of a specified single stranded nucleic acid sequence and allowing selection of those sequences likely to occur in exposed single stranded regions of a folded mRNA. Methods and compositions for designing appropriate oligonucleotides may be found, for example, in U.S. Pat. No. 6,251,588, the contents of which are incorporated herein by reference.

Phosphorothioate antisense oligonucleotides may be used. Modifications of the phosphodiester linkage as well as of the heterocycle or the sugar may provide an increase in efficiency. Phosphorothioate is used to modify the phosphodiester linkage. An N3′-P5′ phosphoramidate linkage has been described as stabilizing oligonucleotides to nucleases and increasing the binding to RNA. A peptide nucleic acid (PNA) linkage is a complete replacement of the ribose and phosphodiester backbone and is stable to nucleases, increases the binding affinity to RNA, and does not allow cleavage by RNAse H. Its basic structure is also amenable to modifications that may allow its optimization as an antisense component. With respect to modifications of the heterocycle, certain heterocycle modifications have proven to augment antisense effects without interfering with RNAse H activity. An example of such modification is C-5 thiazole modification. Finally, modification of the sugar may also be considered. 2′-O-propyl and 2′-methoxyethoxy ribose modifications stabilize oligonucleotides to nucleases in cell culture and in vivo.

Short interfering (si) RNA technology (also known as RNAi) generally involves degradation of an mRNA of a particular sequence induced by double-stranded RNA (dsRNA) that is homologous to that sequence, thereby “interfering” with expression of the corresponding gene. A selected gene may be repressed by introducing a dsRNA which corresponds to all or a substantial part of the mRNA for that gene. Without being held to theory, it is believed that when a long dsRNA is expressed, it is initially processed by a ribonuclease III into shorter dsRNA oligonucleotides of as few as 21 to 22 base pairs in length. Accordingly, siRNA may be effected by introduction or expression of relatively short homologous dsRNAs. Exemplary siRNAs have sense and antisense strands of about 21 nucleotides that form approximately 19 nucleotides of double stranded RNA with overhangs of two nucleotides at each 3′ end.

siRNA has proven to be an effective means of decreasing gene expression in a variety of cell types. siRNA typically decreases expression of a gene to lower levels than that achieved using antisense techniques, and frequently eliminates expression entirely. In mammalian cells, siRNAs are effective at concentrations that are several orders of magnitude below the concentrations typically used in antisense experiments.

The double stranded oligonucleotides used to effect RNAi are specifically less than 30 base pairs in length, for example, about 29, 28, 27, 26, 25, 24, 23, 22, 21, 20, 19, 18, or 17 base pairs or less in length, and contain a segment sufficiently complementary to the target mRNA to allow hybridization to the target mRNA. Optionally, the dsRNA oligonucleotide includes 3′ overhang ends. Exemplary 2-nucleotide 3′ overhangs are composed of ribonucleotide residues of any type and may be composed of 2′-deoxythymidine residues, which lowers the cost of RNA synthesis and may enhance nuclease resistance of siRNAs in the cell culture medium and within transfected cells. Exemplary dsRNAs are synthesized chemically or produced in vitro or in vivo using appropriate expression vectors. Longer RNAs may be transcribed from promoters, such as T7 RNA polymerase promoters, known in the art.

Longer dsRNAs of 50, 75, 100, or even 500 base pairs or more also may be utilized in certain embodiments. Exemplary concentrations of dsRNAs for effecting RNAi are about 0.05 nM, 0.1 nM, 0.5 nM, 1.0 nM, 1.5 nM, 25 nM, or 100 nM, although other concentrations may be utilized depending upon the nature of the cells treated, the gene target and other factors readily identifies by one of ordinary skill in the art.

Compared to siRNA, shRNA offers advantages in silencing longevity and delivery options. Vectors that produce shRNAs, which are processed intracellularly into short duplex RNAs having siRNA-like properties provide a renewable source of a gene-silencing reagent that can mediate persistent gene silencing after stable integration of the vector into the host-cell genome. Furthermore, the core silencing ‘hairpin’ cassette can be readily inserted into retroviral, lentiviral, or adenoviral vectors, facilitating delivery of shRNAs into a broad range of cell types.

A hairpin can be organized in either a left-handed hairpin (i.e., 5′-antisense-loop-sense-3′) or a right-handed hairpin (i.e., 5′-sense-loop-antisense-3′). The shRNA may also contain overhangs at either the 5′ or 3′ end of either the sense strand or the antisense strand, depending upon the organization of the hairpin. If there are any overhangs, they are specifically on the 3′ end of the hairpin and include 1 to 6 bases. The overhangs can be unmodified, or can contain one or more specificity or stabilizing modifications, such as a halogen or O-alkyl modification of the 2′ position, or internucleotide modifications such as phosphorothioate, phosphorodithioate, or methylphosphonate modifications. The overhangs can be ribonucleic acid, deoxyribonucleic acid, or a combination of ribonucleic acid and deoxyribonucleic acid.

Additionally, a hairpin can further comprise a phosphate group on the 5′-most nucleotide. The phosphorylation of the 5′-most nucleotide refers to the presence of one or more phosphate groups attached to the 5′ carbon of the sugar moiety of the 5′-terminal nucleotide. Specifically, there is only one phosphate group on the 5′ end of the region that will form the antisense strand following Dicer processing. In one exemplary embodiment, a right-handed hairpin can include a 5′ end (i.e., the free 5′ end of the sense region) that does not have a 5′ phosphate group, or can have the 5′ carbon of the free 5′-most nucleotide of the sense region being modified in such a way that prevents phosphorylation. This can be achieved by a variety of methods including, but not limited to, addition of a phosphorylation blocking group (e.g., a 5′-O-alkyl group), or elimination of the 5′—OH functional group (e.g., the 5′-most nucleotide is a 5′-deoxy nucleotide). In cases where the hairpin is a left-handed hairpin, preferably the 5′ carbon position of the 5′-most nucleotide is phosphorylated.

Hairpins that have stem lengths longer than 26 base pairs can be processed by Dicer such that some portions are not part of the resulting siRNA that facilitates mRNA degradation. Accordingly the first region, which may include sense nucleotides, and the second region, which may include antisense nucleotides, may also contain a stretch of nucleotides that are complementary (or at least substantially complementary to each other), but are or are not the same as or complementary to the target mRNA. While the stem of the shRNA can include complementary or partially complementary antisense and sense strands exclusive of overhangs, the shRNA can also include the following: (1) the portion of the molecule that is distal to the eventual Dicer cut site contains a region that is substantially complementary/homologous to the target mRNA; and (2) the region of the stem that is proximal to the Dicer cut site (i.e., the region adjacent to the loop) is unrelated or only partially related (e.g., complementary/homologous) to the target mRNA. The nucleotide content of this second region can be chosen based on a number of parameters including but not limited to thermodynamic traits or profiles.

Modified shRNAs can retain the modifications in the post-Dicer processed duplex. In exemplary embodiments, in cases in which the hairpin is a right handed hairpin (e.g., 5′-S-loop-AS-3′) containing 2-6 nucleotide overhangs on the 3′ end of the molecule, 2′-O-methyl modifications can be added to nucleotides at position 2, positions 1 and 2, or positions 1, 2, and 3 at the 5′ end of the hairpin. Also, Dicer processing of hairpins with this configuration can retain the 5′ end of the sense strand intact, thus preserving the pattern of chemical modification in the post-Dicer processed duplex. Presence of a 3′ overhang in this configuration can be particularly advantageous since blunt ended molecules containing the prescribed modification pattern can be further processed by Dicer in such a way that the nucleotides carrying the 2′ modifications are removed. In cases where the 3′ overhang is present/retained, the resulting duplex carrying the sense-modified nucleotides can have highly favorable traits with respect to silencing specificity and functionality. Examples of exemplary modification patterns are described in detail in U.S. Patent Publication No. 20050223427 and International Patent Publication Nos. WO 2004/090105 and WO 2005/078094, the disclosures of each of which are incorporated by reference herein in their entirety.

shRNA may comprise sequences that were selected at random, or according to a rational design selection procedure. For example, rational design algorithms are described in International Patent Publication No. WO 2004/045543 and U.S. Patent Publication No. 20050255487, the disclosures of which are incorporated herein by reference in their entireties. Additionally, it may be desirable to select sequences in whole or in part based on average internal stability profiles (“AISPs”) or regional internal stability profiles (“RISPs”) that may facilitate access or processing by cellular machinery.

Ribozymes are enzymatic RNA molecules capable of catalyzing specific cleavage of mRNA, thus preventing translation. The mechanism of ribozyme action involves sequence specific hybridization of the ribozyme molecule to complementary target RNA, followed by an endonucleolytic cleavage event. The ribozyme molecules specifically include (1) one or more sequences complementary to a target mRNA, and (2) the well-known catalytic sequence responsible for mRNA cleavage or a functionally equivalent sequence (see, e.g., U.S. Pat. No. 5,093,246, which is incorporated herein by reference in its entirety).

While ribozymes that cleave mRNA at site-specific recognition sequences can be used to destroy target mRNAs, hammerhead ribozymes may alternatively be used. Hammerhead ribozymes cleave mRNAs at locations dictated by flanking regions that form complementary base pairs with the target mRNA. Specifically, the target mRNA has the following sequence of two bases: 5′-UG-3′. The construction and production of hammerhead ribozymes is well known in the art and is described more fully in U.S. Pat. No. 5,633,133, the contents of which are incorporated herein by reference.

Gene targeting ribozymes may contain a hybridizing region complementary to two regions of a target mRNA, each of which is at least 5, 6, 7, 8, 9, 10, 11, 12, 13, 14, 15, 16, 17, 18, 19, or 20 contiguous nucleotides (but which need not both be the same length).

Hammerhead ribozyme sequences can be embedded in a stable RNA such as a transfer RNA (tRNA) to increase cleavage efficiency in vivo. In particular, RNA polymerase III-mediated expression of tRNA fusion ribozymes is well known in the art. There are typically a number of potential hammerhead ribozyme cleavage sites within a given target cDNA sequence. Specifically, the ribozyme is engineered so that the cleavage recognition site is located near the 5′ end of the target mRNA—to increase efficiency and minimize the intracellular accumulation of non-functional mRNA transcripts. Furthermore, the use of any cleavage recognition site located in the target sequence encoding different portions of the target mRNA would allow the selective targeting of one or the other target genes.

Ribozymes also include RNA endoribonucleases (“Cech-type ribozymes”) such as the one which occurs naturally in Tetrahymena thermophile, described in International Patent Publication No. WO 88/04300. The Cech-type ribozymes have an eight base pair active site which hybridizes to a target RNA sequence where after cleavage of the target RNA takes place. In one embodiment, Cech-type ribozymes target eight base-pair active site sequences that are present in a target gene or nucleic acid sequence.

Ribozymes can be composed of modified oligonucleotides (e.g., for improved stability, targeting, etc.) and can be chemically synthesized or produced through an expression vector. Because ribozymes, unlike antisense molecules, are catalytic, a lower intracellular concentration is required for efficiency. Additionally, in certain embodiments, a ribozyme may be designed by first identifying a sequence portion sufficient to cause effective knockdown by RNAi. Portions of the same sequence may then be incorporated into a ribozyme.

Alternatively, target gene expression can be reduced by targeting deoxyribonucleotide sequences complementary to the regulatory region of the gene (i.e., the promoter and/or enhancers) to form triple helical structures that prevent transcription of the gene in target cells in the body. Nucleic acid molecules to be used in triple helix formation for the inhibition of transcription are specifically single stranded and composed of deoxyribonucleotides. The base composition of these oligonucleotides should promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in CGC triplets across the three strands in the triplex.

Alternatively, the target sequences that can be targeted for triple helix formation may be increased by creating a so-called “switchback” nucleic acid molecule. Switchback molecules are synthesized in an alternating 5′-3′, 3′-5′ manner, such that they base pair with first one strand of a duplex and then the other, eliminating the necessity for a sizable stretch of either purines or pyrimidines to be present on one strand of a duplex.

In an aspect, a small hairpin RNA is shRNA- HMGB2:

(SEQ ID NO: 8)
CCGGGCTCAACATTAGCTTCAGTATCTCGAGATACTGAAGCTA
ATGTTGAGCTTTTTG

In an aspect, the HMGB2 inhibitor is inflachromene or an analog thereof. Inflachromene (ICM) analogs include those compounds based on modification of the ICM compound at, for example, Region I, Region II, Region III, or a combination thereof in the following scheme.

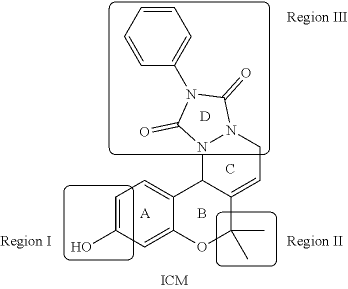

In an embodiment, the ICM analog is a compound of Formula (I)

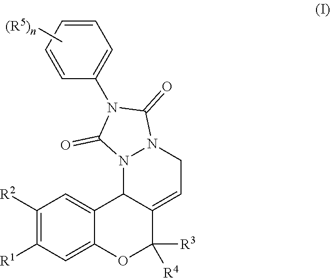

wherein

R¹ and R² are each independently hydrogen, —OH, C₁-C₆ alkyl, C₁-C₆ alkoxy, C₂-C₆ alkenyl, C₂-C₆ alkynyl, C₂-C₆ alkanoyl, C₂-C₆ alkoxycarbonyl, mono- or di-C₁-C₆ alkylcarboxamide, mono- or di-C₁-C₆ alkylamino, aryl, (aryl)C₁-C₆ alkyl, substituted or unsubstituted (aryl)carbonyl, C₃-C₈ cycloalkyl, halogen, C₁-C₆ haloalkyl, C₁-C₆ haloalkoxy, heteroaryl, (heteroaryl)C₁-C₆ alkyl, heterocycloalkyl, —COOH, —CN, —NH₂, —NO₂, (C₁-C₆ alkyl)amido, (C₂-C₆ alkenyl)amido, or (C₂-C₆ alkynyl)amido;

R³ and R⁴ are each independently hydrogen, —OH, C₁-C₆ alkyl, C₁-C₆ alkoxy, C₂-C₆ alkenyl, C₂-C₆ alkynyl, C₂-C₆ alkanoyl, C₂-C₆ alkoxycarbonyl, mono- or di-C₁-C₆ alkylcarboxamide, mono- or di-C₁-C₆ alkylamino, aryl, (aryl)C₁-C₆ alkyl, substituted or unsubstituted (aryl)carbonyl, C₃-C₈ cycloalkyl, halogen, C₁-C₆ haloalkyl, C₁-C₆ haloalkoxy, heteroaryl, (heteroaryl)C₁-C₆ alkyl, heterocycloalkyl, —COOH, —CN, —NH₂, —NO₂, (C₁-C₆ alkyl)amido, (C₂-C₆ alkenyl)amido, (C₂-C₆ alkynyl)amido, or R³ and R⁴ together can form a substituted or unsubstituted C₃-C₆ cycloalkyl, or substituted or unsubstituted C₃-C₆ heterocycloalkyl;

each instance of R⁵ is hydrogen, —OH, C₁-C₆ alkyl, C₁-C₆ alkoxy, C₂-C₆ alkenyl, C₂-C₆ alkynyl, C₂-C₆ alkanoyl, C₂-C₆ alkoxycarbonyl, mono- or di-C₁-C₆ alkylcarboxamide, mono- or di-C₁-C₆ alkylamino, aryl, (aryl)C₁-C₆ alkyl, substituted or unsubstituted (aryl)carbonyl, C₃-C₈ cycloalkyl, halogen, C₁-C₆ haloalkyl, C₁-C₆ haloalkoxy, heteroaryl, (heteroaryl)C₁-C₆ alkyl, heterocycloalkyl, —COOH, —CN, —NH₂, —NO₂, (C₁-C₆ alkyl)amido, (C₂-C₆ alkenyl)amido, or (C₂-C₆ alkynyl)amido; n is 0, 1, 2, or 3; or a pharmaceutically acceptable salt thereof; with the proviso that when R² and R⁵ are both hydrogen then R³, and R⁴ are not —OH, methyl, and methyl, respectively. The groups that are substituted can be substituted with 1, 2, or 3 substituents such as —OH, C₁-C₆ alkyl, C₁-C₆ alkoxy, C₂-C₆ alkenyl, C₂-C₆ alkynyl, C₂-C₆ alkanoyl, C₂-C₆ alkoxycarbonyl, mono- or di-C₁-C₆ alkylcarboxamide, mono- or di-C₁-C₆ alkylamino, aryl, (aryl)C₁-C₆ alkyl, substituted or unsubstituted (aryl)carbonyl, C₃-C₈ cycloalkyl, halogen, C₁-C₆ haloalkyl, C₁-C₆ haloalkoxy, heteroaryl, (heteroaryl)C₁-C₆ alkyl, heterocycloalkyl, —COOH, —CN, —NH₂, —NO₂, (C₁-C₆ alkyl)amido, (C₂-C₆ alkenyl)amido, or (C₂-C₆ alkynyl)amido.

In one embodiment, n is 1 and R⁵ is in the para position. In another embodiment, n is 2 and the R⁵ groups are in the ortho and para positions or in the meta and para positions. In another embodiment, n is 3 and the R⁵ groups are in the ortho and para positions.

In one embodiment, the ICM analog is a compound of Formula (I) wherein R¹ is —OH, R² is hydrogen, R³ is methyl, R⁴ is methyl, and R⁵ is —OH, C₁-C₆ alkyl, C₁-C₆ alkoxy, C₂-C₆ alkenyl, C₂-C₆ alkynyl, C_{z}-C₆ alkanoyl, C₂-C₆ alkoxycarbonyl, mono- or di-C₁-C₆ alkylcarboxamide, mono- or di-C₁-C₆ alkylamino, aryl, (aryl)C₁-C₆ alkyl, substituted or unsubstituted (aryl)carbonyl, C₃-C₈ cycloalkyl, halogen, C₁-C₆ haloalkyl, C₁-C₆ haloalkoxy, heteroaryl, (heteroaryl)C₁-C₆ alkyl, heterocycloalkyl, —COOH, —CN, —NH₂, —NO₂, (C₁-C₆ alkyl)amido, (C₂-C₆ alkenyl)amido, or (C₂-C₆ alkynyl)amido; or a pharmaceutically acceptable salt thereof.

In another embodiment, the ICM analog is a compound of Formula (I) wherein R¹ is —OH, R² is hydrogen, R³ is methyl, R⁴ is methyl, and R⁵ is C₁-C₆ alkyl, or substituted or unsubstituted (aryl)carbonyl, specifically (phenyl)carbonyl. The substituted (aryl)carbonyl can be substituted with 1, 2, or 3 substituents such as —OH, C₁-C₆ alkyl, C₁-C₆ alkoxy, C₂-C₆ alkenyl, C₂-C₆ alkynyl, C₂-C₆ alkanoyl, C₂-C₆ alkoxycarbonyl, mono- or di-C₁-C₆ alkylcarboxamide, mono- or di-C₁-C₆ alkylamino, aryl, (aryl)C₁-C₆ alkyl, substituted or unsubstituted (aryl)carbonyl, C₃-C₈ cycloalkyl, halogen, C₁-C₆ haloalkyl, C₁-C₆ haloalkoxy, heteroaryl, (heteroaryl)C₁-C₆ alkyl, heterocycloalkyl, —COOH, —CN, or —NO₂, (C₁-C₆ alkyl)amido, (C₂-C₆ alkenyl)amido, or (C₂-C₆ alkynyl)amido.

In another embodiment, the ICM analog is a compound of Formula (I) wherein R¹ is —OH, R² is hydrogen, R⁵ is hydrogen, and R³ and R⁴ are each independently hydrogen, —OH, C₁-C₆ alkyl, C₁-C₆ alkoxy, C₂-C₆ alkenyl, C₂-C₆ alkynyl, C₂-C₆ alkanoyl, C₂-C₆ alkoxycarbonyl, mono- or di-C₁-C₆ alkylcarboxamide, mono- or di-C₁-C₆ alkylamino, aryl, (aryl)C₁-C₆ alkyl, substituted or unsubstituted (aryl)carbonyl, C₃-C₈ cycloalkyl, halogen, C₁-C₆ haloalkyl, C₁-C₆ haloalkoxy, heteroaryl, (heteroaryl)C₁-C₆ alkyl, heterocycloalkyl, —COOH, —CN, —NH₂, —NO₂, (C₁-C₆ alkyl)amido, (C₂-C₆ alkenyl)amido, (C₂-C₆ alkynyl)amido, or R³ and R⁴ together can form a substituted or unsubstituted C₃-C₆ cycloalkyl, or substituted or unsubstituted C₃-C₆ heterocycloalkyl; with the proviso that at least one of R³ and R⁴ is other than hydrogen.

In another embodiment, the ICM analog is a compound of Formula (I) wherein R¹ is —OH, R² is hydrogen, R⁵ is hydrogen, and R³ and R⁴ together form a substituted or unsubstituted C₃-C₆ heterocycloalkyl, specifically a substituted piperidine.

In an embodiment, the ICM analog is a compound of Formula (I) wherein R¹ is C₁-C₆ alkyl, C₁-C₆ alkoxy, C₂-C₆ alkenyl, C₂-C₆ alkynyl, C₂-C₆ alkanoyl, C₂-C₆ alkoxycarbonyl, mono- or di-C₁-C₆ alkylcarboxamide, mono- or di-C₁-C₆ alkylamino, aryl, (aryl)C₁-C₆ alkyl, substituted or unsubstituted (aryl)carbonyl, C₃-C₈ cycloalkyl, halogen, C₁-C₆ haloalkyl, C₁-C₆ haloalkoxy, heteroaryl, (heteroaryl)C₁-C₆ alkyl, heterocycloalkyl, —COOH, —CN, —NH₂, —NO₂, (C₁-C₆ alkyl)amido, (C₂-C₆ alkenyl)amido, or (C₂-C₆ alkynyl)amido; R² is hydrogen; R⁵ is hydrogen; and R³ and R⁴ are each methyl.

In another embodiment, the ICM analog is a compound of Formula (I) wherein R¹ is C₁-C₆ alkoxy or C₁-C₆ haloalkoxy; R² is hydrogen; R⁵ is hydrogen; and R³ and R⁴ are each methyl.

In an embodiment, the ICM analog is a compound of Formula (I) wherein R² is —OH, C₁-C₆ alkyl, C₁-C₆ alkoxy, C₂-C₆ alkenyl, C₂-C₆ alkynyl, C₂-C₆ alkanoyl, C₂-C₆ alkoxycarbonyl, mono- or di-C₁-C₆ alkylcarboxamide, mono- or di-C₁-C₆ alkylamino, aryl, (aryl)C₁-C₆ alkyl, substituted or unsubstituted (aryl)carbonyl, C₃-C₈ cycloalkyl, halogen, C₁-C₆ haloalkyl, C₁-C₆ haloalkoxy, heteroaryl, (heteroaryl)C₁-C₆ alkyl, heterocycloalkyl, —COOH, —CN, —NH₂, —NO₂, (C₁-C₆ alkyl)amido, (C₂-C₆ alkenyl)amido, or (C₂-C₆ alkynyl)amido; R¹ is —OH; R⁵ is hydrogen; and R³ and R⁴ are each methyl.

In another embodiment, the ICM analog is a compound of Formula (I) wherein R² is C₁-C₆ alkoxy or C₁-C₆ haloalkoxy; R¹ is —OH; R⁵ is hydrogen; and R³ and R⁴ are each methyl.

In certain situations, the compounds of Formula I may contain one or more asymmetric elements such as stereogenic centers, stereogenic axes and the like, e.g., asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, it should be understood that all of the optical isomers and mixtures thereof are encompassed. In addition, compounds with double bonds may occur in Z- and E-forms, with all isomeric forms of the compounds being included in the present disclosure. In these situations, the single enantiomers, i.e., optically active forms, can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column.

The term “substituted”, as used herein, means that any one or more hydrogens on the designated atom or group is replaced with a selection from the indicated group, provided that the designated atom's normal valence is not exceeded. When a substituent is oxo (i.e., ═O), then 2 hydrogens on the atom are replaced. When aromatic moieties are substituted by an oxo group, the aromatic ring is replaced by the corresponding partially unsaturated ring. For example a pyridyl group substituted by oxo is a pyridone.

Combinations of sub stituents and/or variables are permissible only if such combinations result in stable compounds or useful synthetic intermediates. A stable compound or stable structure is meant to imply a compound that is sufficiently robust to survive isolation from a reaction mixture, and subsequent formulation into an effective therapeutic agent.

A dash (“-”) that is not between two letters or symbols is used to indicate a point of attachment for a substituent. For example, —COOH is attached through the carbon atom.

As used herein, “alkyl” is intended to include both branched and straight-chain saturated aliphatic hydrocarbon groups, having the specified number of carbon atoms. Thus, the term C₁- C₆ alkyl as used herein includes alkyl groups having from 1 to about 6 carbon atoms. When C₀-C₄ alkyl is used herein in conjunction with another group, for example, phenylC₀-C₄ alkyl, the indicated group, in this case phenyl, is either directly bound by a single covalent bond (C₀), or attached by an alkyl chain having the specified number of carbon atoms, in this case from 1 to about 2 carbon atoms. Examples of alkyl include, but are not limited to, methyl, ethyl, n-propyl, isopropyl, n-butyl, t-butyl, n-pentyl, and sec-pentyl.

“Alkenyl” as used herein, indicates hydrocarbon chains of either a straight or branched configuration comprising one or more unsaturated carbon- carbon bonds, which may occur in any stable point along the chain, such as ethenyl and propenyl.

“Alkynyl” as used herein, indicates hydrocarbon chains of either a straight or branched configuration comprising one or more triple carbon-carbon bonds that may occur in any stable point along the chain, such as ethynyl and propynyl.

“Alkoxy” represents an alkyl group as defined above with the indicated number of carbon atoms attached through an oxygen bridge. Examples of alkoxy include, but are not limited to, methoxy, ethoxy, n-propoxy, propoxy, n-butoxy,2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy.

“Alkanoyl” indicates an alkyl group as defined above, attached through a keto (—(C═O)—) bridge. Alkanoyl groups have the indicated number of carbon atoms, with the carbon of the keto group being included in the numbered carbon atoms. For example a C₂alkanoyl group is an acetyl group having the formula CH₃(C═O)—.

The term “alkoxycarbonyl” indicates an alkoxy group, as defined above, having the indicated number of carbon atoms, attached through a keto linkage. The carbon of the keto linker is not included in the numbering, thus a C₂alkoxycarbonyl has the formula CH₃CH₂O(C═O)—.

The term “alkylcarboxamide” indicates an alkyl group, as defined above, having the indicated number of carbon atoms, attached through a carboxamide linkage, i.e., a —CONH₂ linkage, where one or both of the amino hydrogens is replaced by an alkyl group. Alkylcarboxamide groups may be mono- or di-alkylcarboxamide groups, such an ethylcarboxamide or dimethylcarboxamide.

The term “(alkyl)amido”, “(alkenyl)amido”, “(alkynyl)amido” indicates an alkyl, alkenyl, or alkynyl group as defined above having the indicated number of carbon atoms, attached through an amide linkage, i.e., a (alkyl)CONH—, (alkenyl)CONH—, or (alkynyl)CONH— linkage.

As used herein, the term “mono- or di-alkylamino” indicates secondary or tertiary alkyl amino groups, wherein the alkyl groups are as defined above and have the indicated number of carbon atoms. The point of attachment of the alkylamino group is on the nitrogen. Examples of mono- and di-alkylamino groups include ethylamino, dimethylamino, and methyl-propyl-amino.

As used herein, the term “aryl” indicates aromatic groups containing only carbon in the aromatic ring or rings. Such aromatic groups may be further substituted with carbon or non-carbon atoms or groups. Typical aryl groups contain 1 to 3 separate, fused, or pendant rings and from 6 to about 18 ring atoms, without heteroatoms as ring members. Where indicated aryl groups may be substituted. Such substitution may include fusion to a 5 to 7-membered saturated cyclic group that optionally contains 1 or 2 heteroatoms independently chosen from N, O, and S, to form, for example, a 3,4-methylenedioxy-phenyl group. Aryl groups include, for example, phenyl, naphthyl, including 1-naphthyl and 2-naphthyl, and bi-phenyl.

In the term “(aryl)alkyl”, aryl and alkyl are as defined above, and the point of attachment is on the alkyl group. This term encompasses, but is not limited to, benzyl, phenylethyl, and piperonyl. Likewise, in the term (aryl)carbhydryl, aryl and carbhydryl are as defined above and the point of attachment is on the carbhydryl group, for example a phenylpropen-1-yl group.

“(Aryl)carbonyl” as used herein, includes the term aryl as defined above, and the point of attachment is through a keto (—(C═O)—) bridge. For example, (phenyl)carbonyl has the formula Ph(C═O)—. The aryl can be unsubstituted or substituted.

“Carbhydryl” as used herein, includes both branched and straight-chain hydrocarbon groups, which are saturated or unsaturated, having the specified number of carbon atoms.

“Cycloalkyl” as used herein, indicates saturated hydrocarbon ring groups, having the specified number of carbon atoms, usually from 3 to about 8 ring carbon atoms, or from 3 to about 7 carbon atoms. Examples of cycloalkyl groups include cyclopropyl, cyclobutyl, cyclopentyl, or cyclohexyl as well as bridged or caged saturated ring groups such as norbornane or adamantane.

“Haloalkyl” indicates both branched and straight-chain saturated aliphatic hydrocarbon groups having the specified number of carbon atoms, substituted with 1 or more halogen atoms, generally up to the maximum allowable number of halogen atoms. Examples of haloalkyl include, but are not limited to, trifluoromethyl, difluoromethyl, 2-fluoroethyl, and penta-fluoroethyl.

“Haloalkoxy” indicates a haloalkyl group as defined above attached through an oxygen bridge.

“Halo” or “halogen” as used herein refers to fluoro, chloro, bromo, or iodo.

As used herein, “heteroaryl” indicates a stable 5- to 7-membered monocyclic or 7-to 10- membered bicyclic heterocyclic ring which contains at least 1 aromatic ring that contains from 1 to 4, or preferably from 1 to 3, heteroatoms chosen from N, O, and S, with remaining ring atoms being carbon. When the total number of S and O atoms in the heteroaryl group exceeds 1, these heteroatoms are not adjacent to one another. It is preferred that the total number of S and O atoms in the heteroaryl group is not more than 2. Examples of heteroaryl groups include, but are not limited to, pyridyl, indolyl, pyrimidinyl, pyridizinyl, pyrazinyl, imidazolyl, oxazolyl, furanyl, thiophenyl, thiazolyl, triazolyl, tetrazolyl, isoxazolyl, quinolinyl, pyrrolyl, pyrazolyl, and 5,6,7,8-tetrahydroisoquinoline. In the term “heteroarylalkyl,” heteroaryl and alkyl are as defined above, and the point of attachment is on the alkyl group. This term encompasses, but is not limited to, pyridylmethyl, thiophenylmethyl, and pyrrolyl(1-ethyl).

The term “heterocycloalkyl” is used to indicate saturated cyclic groups containing from 1 to about 3 heteroatoms chosen from N, O, and S, with remaining ring atoms being carbon. Heterocycloalkyl groups have from 3 to about 8 ring atoms, and more typically have from 5 to 7 ring atoms. A C₂-C₇heterocycloalkyl group contains from 2 to about 7 carbon ring atoms and at least one ring atom chosen from N, O, and S. Examples of heterocycloalkyl groups include morpholinyl, piperazinyl, piperidinyl, and pyrrolidinyl groups.

“Pharmaceutically acceptable salts” includes derivatives of the disclosed compounds wherein the parent compound is modified by making an acid or base salt thereof, and further refers to pharmaceutically acceptable solvates of such compounds and such salts. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional salts and the quaternary ammonium salts of the parent compound formed, for example, from inorganic or organic acids. For example, conventional acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like. The pharmaceutically acceptable salts of the present invention can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable.

In an aspect, the HMGB2 inhibitor is administered in the form of a liposome particle, specifically an anionic liposome particle. As used herein, the term “liposome” refers to a vesicle composed of amphiphilic lipids arranged in at least one bilayer, e.g., one bilayer or a plurality of bilayers. Liposomes include unilamellar and multilamellar vesicles that have a membrane formed from a lipophilic material and an aqueous interior. Liposomes typically have diameters of 25 nm to several microns, specifically 25 to 500 nm.

In a specific aspect, the HMGB2 inhibitor is administered in the form of an anionic liposome particle formulated to target stellate cells, for example, liposome particles containing a targeting agent such as Vitamin A and optionally a small molecule HMGB2 inhibitor. When the liposome particles are assembled in the presence of Vitamin A and the optional small molecule HMGB2 inhibitor, the Vitamin A and the optional small molecule HMGB2 inhibitor become incorporated into the bilayer. Alternatively, the liposomes may be formed using cationic lipids and neutral lipids. Cationic lipids can directly interact with the nucleic acid therapeutics via charge interactions to form a liposome complex. The anionic lipids for complexes with the nucleic acids therapeutics via an intermediary positively charged species such as Ca²⁺.

In a typical method of making a liposome particle, an ethanol injection method is used in which a lipid or lipids are dissolved in ethanol and the solution is injected into an aqueous phase. Under conditions such as low lipid concentration, the result is a dispersion of liposomes having controllable particle sizes and particle size distributions. As an alternative, liposome particles can be formed using a co-axial turbulent jet in co-flow which can be used to produce liposome particles having a controllable mean particle size. In the method, both the aqueous and the ethanol (with dissolved lipid and optionally Vitamin A and/or a small molecule HMGB2 inhibitor) streams flow in the same direction (i.e., co-flow). The flows are then mixed just prior to turbulent jet formation. By altering the injection port dimensions and flow rates, unilamellar, monodisperse liposome particles can be produced. The mean liposome size is dependent upon the Reynolds number of the mixed ethanol/aqueous phase and independent of the flow velocity ratio.

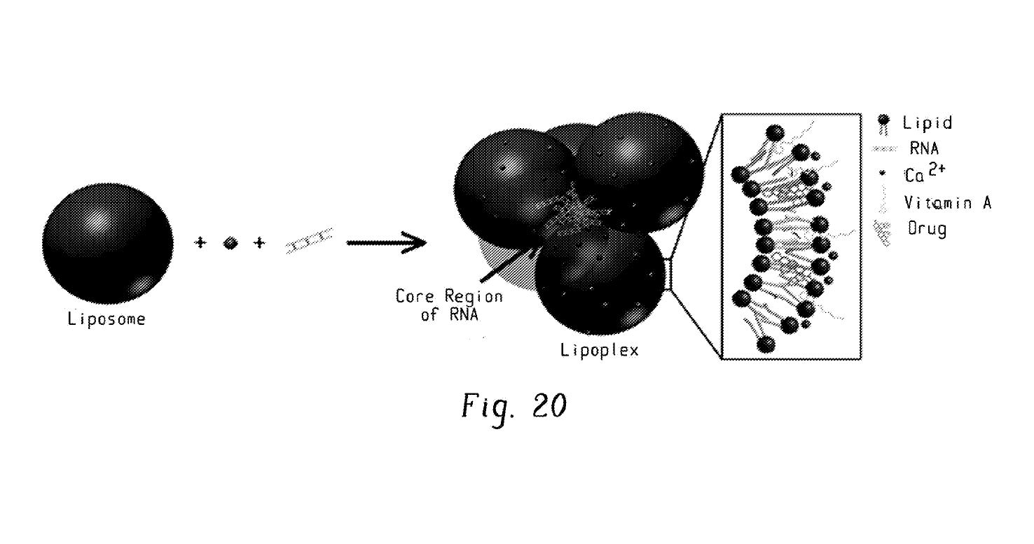

Once the anionic liposome particles are produced, they can be coated with a positively-charged species such as Ca²⁺, Mg²⁺, chitosan, and the like, to provide adhesion of a nucleic acid therapeutic agent such as an interfering RNA or a micro RNA. When the coated liposome particles are contacted with the RNA therapeutic, the RNA is complexed between the liposome particles forming a liposome complex. (See, FIG. 20)

Exemplary anionic and neutral lipids for inclusion into anionic liposome particles include di stearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof.

In an aspect, an anionic liposome comprises DPPG (1,2-dipalmitoyl-sn-glycero-3-phospho-(1′-rac-glycerol)), cholesterol and a fusogenic helper lipid, DOPE (1,2-dioleoyl-sn-glycero-3- phosphoethanolamine).

Further included are pharmaceutical compositions comprising a pharmaceutically acceptable excipient and an inhibitor of HMGB2, optionally in the form of a liposome particle.

As used herein, “pharmaceutical composition” means therapeutically effective amounts of an inhibitor of HMGB2together with a pharmaceutically acceptable excipient, such as diluents, preservatives, solubilizers, emulsifiers, and adjuvants. As used herein “pharmaceutically acceptable excipients” are well known to those skilled in the art.

Tablets and capsules for oral administration may be in unit dose form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavoring or coloring agents.

The active ingredient may also be administered parenterally in a sterile medium, either subcutaneously, or intravenously, or intramuscularly, or intrasternally, or by infusion techniques, in the form of sterile injectable aqueous or oleaginous suspensions. Depending on the vehicle and concentration used, the drug can either be suspended or dissolved in the vehicle. Advantageously, adjuvants such as a local anaesthetic, preservative and buffering agents can be dissolved in the vehicle.

Pharmaceutical compositions may conveniently be presented in unit dosage form and may be prepared by any of the methods well known in the art of pharmacy. The term “unit dosage” or “unit dose” means a predetermined amount of the active ingredient sufficient to be effective for treating an indicated activity or condition. Making each type of pharmaceutical composition includes the step of bringing the active compound into association with a carrier and one or more optional accessory ingredients. In general, the formulations are prepared by uniformly and intimately bringing the active compound into association with a liquid or solid carrier and then, if necessary, shaping the product into the desired unit dosage form.

The invention is further illustrated by the following non-limiting examples.

### EXAMPLES

### Methods

Animal Care: All animal care and experimental procedures were approved by the Institutional Animal Care and Use Committee at the University of Connecticut. Shp^{-/-} mice were described previously. Hmgb2^{-/-} mice were obtained from Dr. Stella Tsirka at Stony Brook University. C57BL/6J mice were purchased from Jackson Laboratory. All mice were maintained on a 12-h light/dark cycle with free access to food and water unless otherwise indicated. For CCl₄ treatment, CCl4 (Sigma) was dissolved in corn oil and administered to mice at a dose of 850 μl/kg BW by intraperitoneal injection (IP) twice a week for 6 or 10 weeks with or without ICM. Age-matched control mice were treated with a corn oil vehicle. For CCl₄+ICM treatment, mice were treated with CCl4 for 2 weeks and then ICM (Department of Pharmaceutical Sciences at UConn) was dissolved in PEG (Sigma) and treated with mice (10 mg/kg) every day for 4 or 8 weeks in combination with CCl₄. To induce overexpression of HMGB2 in liver, AAV8-Hmgb2 or a control AAV8-GFP was given to mice by tail vein injection at a dose of 5×10¹⁰ GC. To knockdown HMGB2 in mouse livers, AAV-shHmgb2 or a control AAV-shGFP was delivered to mice at a dose of 2×10¹⁰ GC by tail vein injection. One week later, mice injected with AAVs were treated with CCl₄ or CCl₄ +ICM as described above and sacrificed. Livers were removed and snap-frozen in liquid nitrogen for RNA and protein analysis. For the collection of mouse fetal livers, pregnant female mice were sacrificed at E12.5, E15, and E18.5 and livers were removed from the embryos. Additionally, post-natal mouse livers were harvested at postnatal day P1 and P60.

RNA Isolation and Analysis: RNA was isolated using TRIzol® (Thermo Fisher) according to the manufacturer's instructions. For quantitative real time RT-PCR (q-PCR), RNA was reverse transcribed using iScript™ cDNA Synthesis Kit (Bio-Rad). The resultant cDNA was amplified and quantified using SsoAdvance™ Universal SYBR® Green Supermix (Bio-Rad) in CFX384 Touch™ Real-Time PCR Detection System (BioRad). qPCR was performed in triplicate for each sample and repeated at three times. Ct values were used to calculate the relative expression level normalized to the levels of Hprt1 or beta-actin expression.

Western Blotting: Total protein lysates were prepared from livers and cells in lysis buffer containing 50 mM Tris, pH 8.0, 1% Nonidet™ P-40, 150 mM NaCl, 0.5% sodium deoxycholate, 0.1% SDS with protease inhibitors (Thermo Fisher Scientific) and subjected to immunoblotting. Nuclear and cytoplasmic subcellular fractions were prepared using NE-PER nuclear and cytoplasmic extraction reagents (Pierce) according to the manufacturer's instructions. The blot was incubated with a primary antibody, followed by a secondary antibody conjugated to horseradish peroxidase. ECL kit (Pierce) was used to detect the reactivity. The following antibodies were obtained from Cell Signaling Technology: HMGB2, HMGB1, AKT, p-AKT, mTOR, p-mTOR, ERK1/2, pERK1/2, Raptor, Rictor, p53, p21, p-Bad, Smad4, p-Smad2/3, Lamin A/C, a-SMA, MMP2, MMP9, TIMP1, RhoA, Rho-GTP, Cdc42, Rac1/2/3. Anti-HA antibody and anti-beta actin antibody were purchased from Sigma and Santa Cruz Biotechnology, respectively.

Co-immunoprecipitation experiments: LX-2 cells were transfected with a vector expressing HA-CBP with X-treineGENE™ transfection reagent (Sigma). 24 hours later, cells were treated with CCl₄ and ICM for 24 hours. Cells then washed 2 times with cold phosphate buffered saline (PBS), lysed in buffer (50 mM Tris HCl, pH 7.4, 150 mM NaCl, 1 mM EDTA, and 1% TRITON X-1.00, and protease inhibitor). Protein extracts were incubated with anti-phosphoserin antibody or anti-acetylated lysin antibody (Cell signaling technology) overnight at 4° C. Protein A/G beads were added to the supernatants and incubated for 4 hours at 4° C. The beads were washed five times in NP-40 lysis buffer. Immune complexes were eluted by SDS—sample buffer, resolved on 10% SDS-PAGE and proceeded for immunoblotting with the indicated antibody.

Histology: Livers were fixed in 10% neutral buffered formalin and embedded in paraffin. Liver blocks were cut at 5 μm and the sections were stained with hematoxylin and eosin (H&E) and Masson's trichrome according to standard procedures.

Primary Mouse Hepatic Stellate Cells (HSCs) isolation and in vitro activation: Primary mouse HSCs were isolated from 8 to 12 weeks old wild type or Hmgb2^{-/-} mice, as described in the art with modifications. Briefly, mice were anesthetized with ketamine/xylazine mixture. The liver was perfused with Hank's balanced salt solution (HBSS) supplemented with 0.5 mM EGTA through the cannulated portal vein, followed by perfusion with HBSS supplemented with 0.5 mg/ml pronase (Sigma) and HBSS with 0.14 mg/ml collagenase (Sigma). The liver was dissected out from the mouse and dissociated cells were dispersed gently in HBSS. Primary HSC was isolated by three-layer discontinuous density gradient centrifugation with 25% and 50% percoll (Sigma) and plated on non-coated culture dishes in DMEM supplemented with 10% FBS, 100 IU/ml penicillin, and 100 μg/ml streptomycin. For culture-activated HSC studies, the medium of primary HSCs was replaced every day up to 7 days. For PDGF treatment, primary HSC cells were serum starved for 24 hr and then treated with PDFG for 3 or 5 days.

Immunofluorescence: Primary mouse HSCs were plated on cover slips and culture-activated, fixed with 4% paraformaldehyde for 15 min, blocked in PBS containing 5% normal serum and 0.3% Triton™ X-100 for 60 min. Cells were incubated with a primary antibody for HMGB2 or α-SMA in PBS containing 1% BSA, 0.3% Triton™ X-100 overnight at 4° C. After five washes with PBS, cells were incubated a secondary antibody for goat anti-rabbit Alexa Fluor® 594 conjugate (Thermo Fisher) or goat anti-mouse Alexa Fluor® 488 conjugate (Thermo Fisher). Slides were washed with PBS and mounted with mounting media containing DAPI.

Cell Lines The LX-2 human stellate cell line was maintained in Dulbecco's Modified Eagle's Medium (DMEM) supplemented with 2% fetal bovine serum (FBS), 100 IU/ml penicillin, and 100 μg/ml streptomycin. Human hepatocellular carcinoma (HCC) MHCC97H cells were maintained in DMEM supplemented with 10% FBS, 100 IU/ml penicillin, and 100 μg/ml streptomycin, 1 mM HEPES, 1 mM sodium pyruvate, 2 mM L-glutamine. Tumor-initiating (TIC) cells were obtained from Dr. Hidekazu Tsukamoto at University of Southern California and were maintained in 1:1 DMEM:F12 supplement with 100 U/ml penicillin and 100 μg/ml streptomycin, 2 mM glutamine, 20 ng/ml mouse EGF, 10 nM dexamethasone, 1× embryomax nucleotides. All the cells were maintained at 37° C. humidified atmosphere with 5% CO₂. TIC cells were infected with Hmgb2 shRNA lentivirus for 48 hr, and treated with 500 ng/ml puromycin for 2 weeks. The stable cells were used for migration and invasion assay, as well as western blot.

Cell Migration and Invasion: Cell migration and invasion assays were performed as described in the art. Briefly, control TIC cells or TIC cells with a stable knockdown of HMGB2 were infected with AAV8-Null or AAV-8-HMGB2 for 48 hr. The cells were then serum starved for 24 hr and 5×10⁴ cells were seeded on transwell inserts with 8 μm pore size (Culterx® 96 well cell migration assay, cat# 3465-095-K). Cells were allowed to migrate for 16 hr. Non-migrating cells were removed from the insert with a cotton swab. The migrated cells were fixed for 10 min (3.7% v/v formaldehyde in PBS), and stained with 0.1% crystal violet for 15 min. Cells were washed with PBS and pictures were taken with Microfire/Qcam CCD Olympus 1×81 microscope. Cells stained with crystal violet were counted and then lysed with 1% SDS for 30 min and the absorbance was measured at 570 nm. Cell invasion assays were performed similarly to migration assays, except that the cells were seeded on pre-coated inserts with BME solution (Cultrex® 96 well BME cell invasion Assay, cat# 3455-096-K). Invaded cells were fixed, stained and the absorbance was measured as in the migration assay.

ELISA:The levels of HMGB2 in serum samples from mice treated with CCl₄ and from patients who were associated with liver diseases were determined using ELISA kits (LifeSpan BioSciences) according to the manufacturer's instructions.

ALT and AST: Alanine transaminase (ALT) and Aspartate aminotransferase (AST) were measured by Infinity ALT (GPT) Liquid Stable Reagent and AST (GOT) Liquid Stable Reagent according to the manufacturer's instructions (Thermo Scientific).

Statistical Analysis: Data were expressed as the mean±standard error of the mean (SEM). All data are representative of at least three independent experiments. Statistical analysis was carried out using Student's t-test and one-way ANOVA. p<0.05 was considered statistically significant.

Cell culture, Cell transfection:HEK293T, Hela, Hep3B and MHCC97H cells were cultured and transfected using X-tremeGENE™ HP DNA transfection reagent (Roche) as described previously. CGR8 ES cells were cultured on 0.1% gelatin coated tissue-culture dish in knockout DMEM medium containing 15% ES qualified-Fetal Bovine Serum, 2 mM L-glutamin, 0.1 mM NEAA, 0.1 mM β-mercaptoethanol, 50 μg/ml penicillin-streptomycin and 10 ng/ml Leukemia inhibitory factor (LIF). TIC cells were cultured in DMEM-F12 medium containing Embryomax nucleotides, 2 mM L-glutamin, 0.1 mM NEAA, 20 ng/ml mouse EGF, 50 μg/ml penicillin-streptomycin and 100 nM dexamethasone.

ES cells EB formation and TICs spheroid assay: CGR8 ES cells were trypsinized into single cells and cultured in ES medium devoid of LIF in hanging drops (900 cells per EB) for 2 d and transferred to ultralow attachment plate for a further 2 d suspension culture. Then, EBs were plated onto gelatin coated tissue-culture plate for further 7 d spontaneously differentiation.

TIC cells were trypsinized into single cells and 1000 cells were suspended in serum free TIC medium with 1% methylcellulose supplemented with B27, 20 ng/ml EGF and 20 ng/ml FGF in each well of 96-well ultralow attachment plate.

Immunoprecipitation and Western Blotting:CGR8 cell lysates were harvested by Myc lysis buffer (138 mM NaCl, 20 mM Tris-HCl (pH 8.0), 1% Nonidet™ P-40), and anti-OCT4 antibody or rabbit IgG were used to immunoprecipitate OCT4 by incubating with protein A/G-Magnetic Beads (Thermo Scientific) at 4° C. for 4 h. After extensive washes, immunoprecipitated proteins were separated by SDS-PAGE, transferred onto PVDF membrane, immunostained with anti-HMGB2 antibody, and finally detected by horseradish peroxidase-conjugated secondary antibodies and visualized by chemiluminescence (Pierce).

Luciferase reporter assay: 48 h after transfection with luciferase reporter plasmid and Renilla plasmid, cells were harvested and analyzed with the Dual-Luciferase reporter assay system (Promega) as described. All assays were done in triplicates, and all values were normalized for transfection efficiency against Renilla luciferase activities.

### Example 1

### HMGB2 Expression is Markedly Induced in Fibrotic and Cirrhotic Liver in Mice and Humans

FIG. 1 shows HMGB2 expression is highly induced in human fibrotic and cirrhotic livers as determined by qPCR analysis. qPCR of Hmgb2 mRNA in human liver samples from fibrosis, steatosis, NASH cirrhosis, alcohol cirrhosis, and HCV cirrhosis patients was performed. It is also induced in mouse models of liver fibrosis.

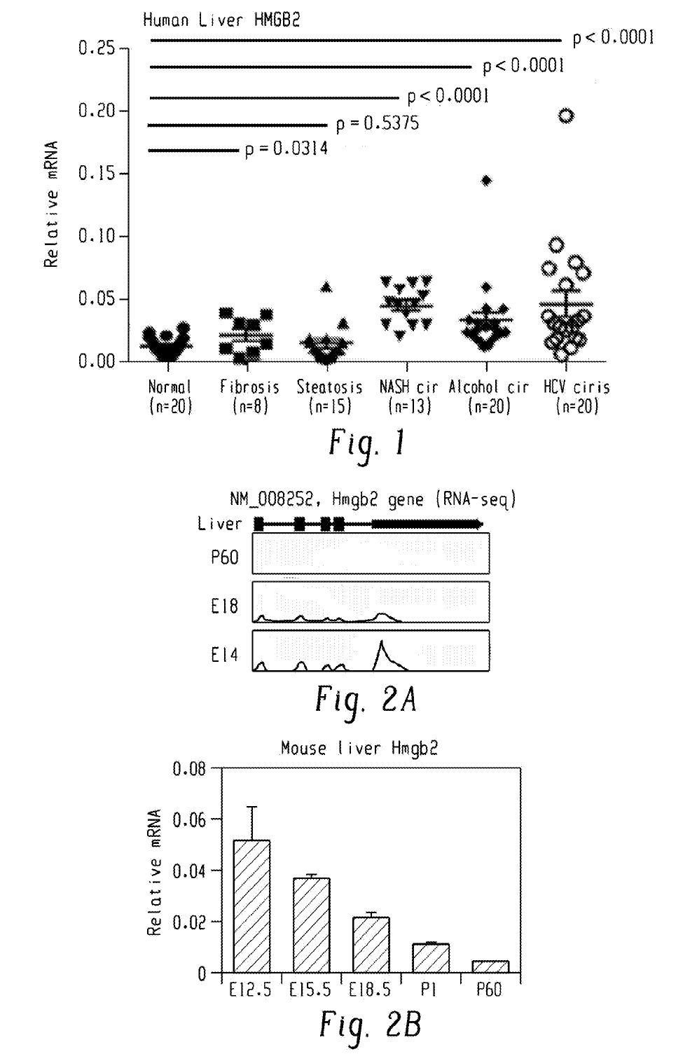

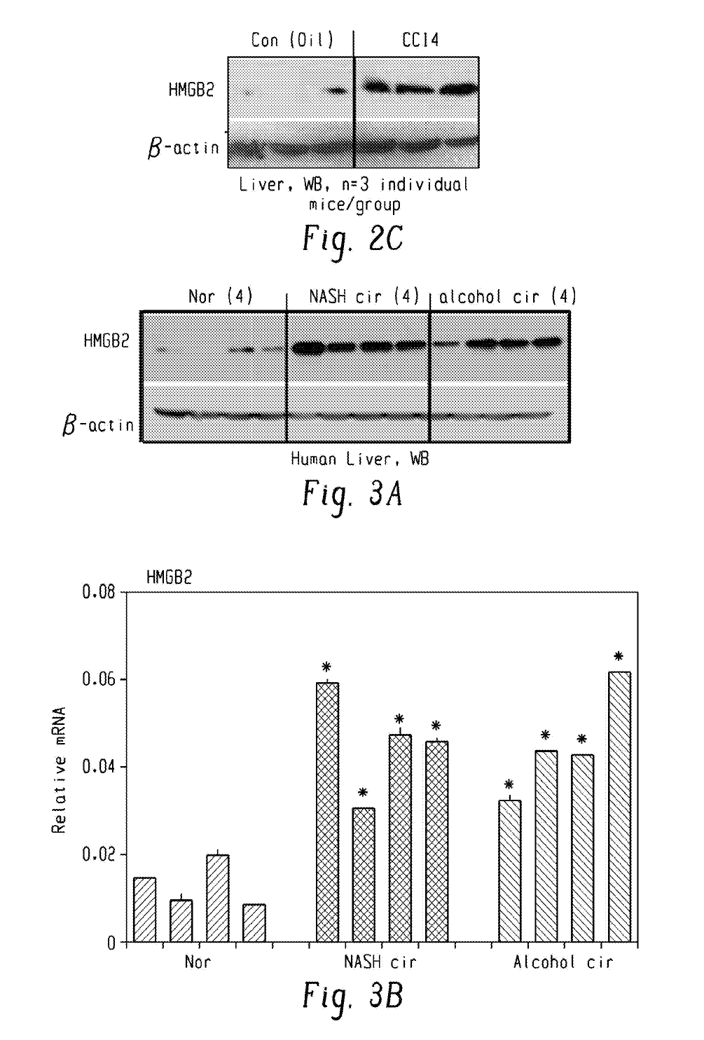

Hepatic HMGB2 expression was high during the embryonic stage but its level drastically decreased in adult liver, as revealed by RNA-seq (FIG. 2A) and validated by qPCR (FIG. 2B). However, the HMGB2 gene was reactivated in CCl₄-induced fibrotic liver in mice (FIG. 2C). Immunohistochemistry was also performed in human liver samples from fibrosis, steatosis, NASH cirrhosis, alcohol cirrhosis, and HCV cirrhosis patients (data not shown). Standard methods were used for qPCR and Western blot.

A Western blot (WB) revealed the induction of HMGB2 protein in human cirrhotic liver (FIG. 3A), which corresponded with its mRNA level (FIG. 3B). The results suggest a regulatory role of HMGB2 in liver fibrosis or cirrhosis. Standard methods were used for qPCR and Western blot.

### Example 2

### Overexpression of HMGB2 Enhances CCl4-Induced Inflammation

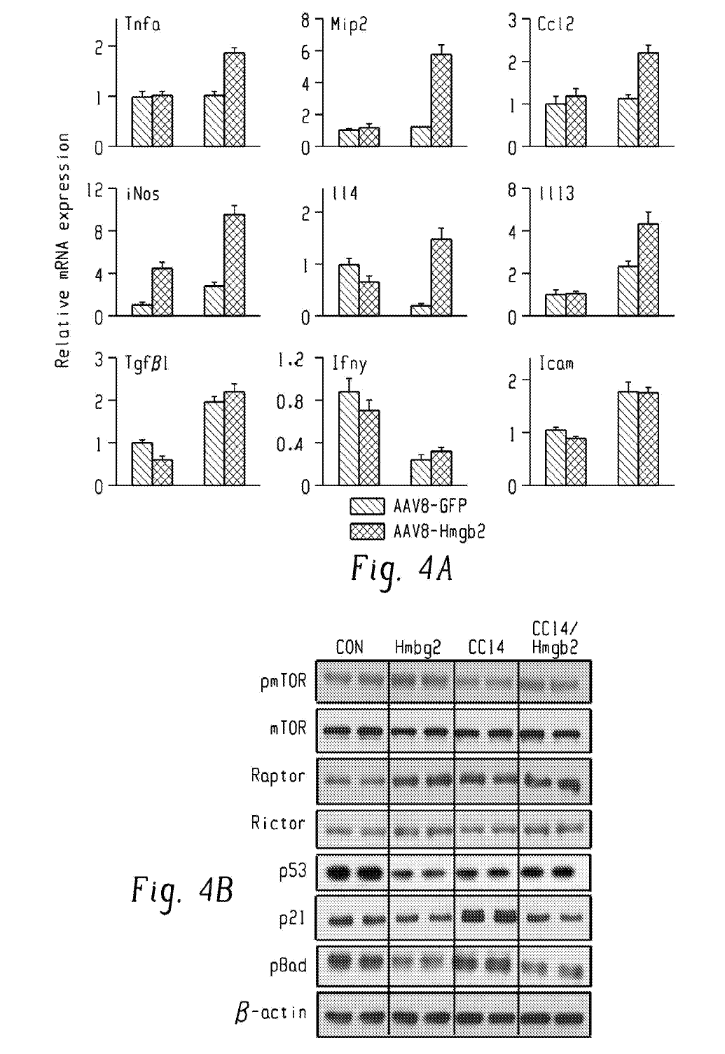

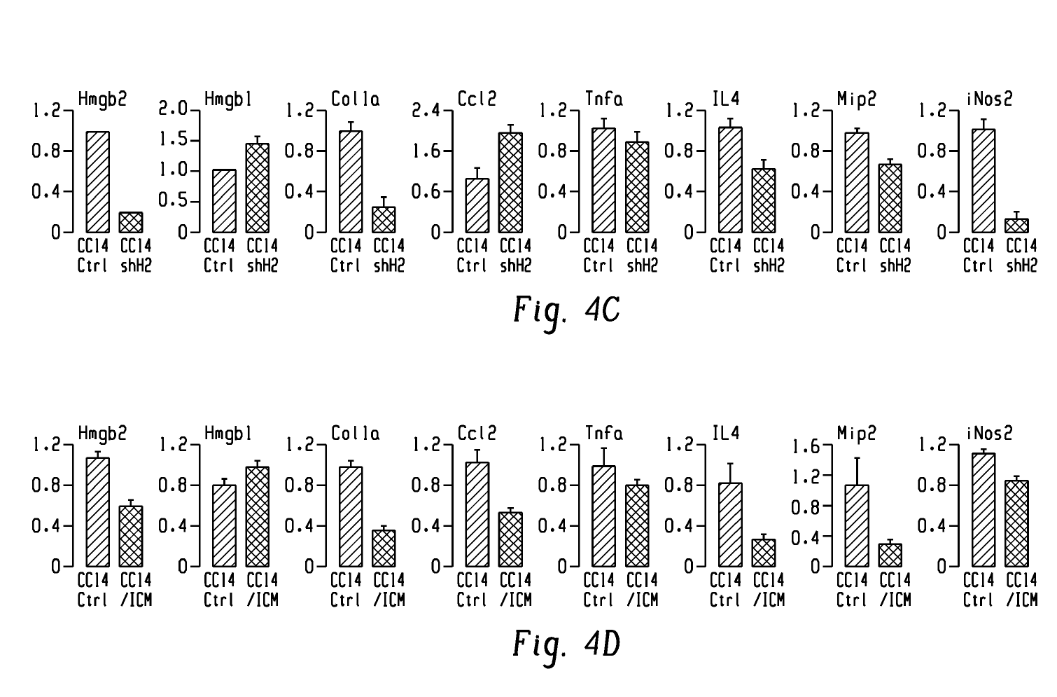

FIG. 4 shows that overexpression of HMGB2 enhances CCl4-induced inflammation (A), increases mTOR pathway and decrease p53 pathway (B). Knockdown HMGB2 by shRNA (C) or ICM (D) decrease inflammatory response.

For CCl₄ treatment, CCl₄ (Sigma) was dissolved in corn oil and administered to mice at a dose of 850 □l/kg BW by intraperitoneal injection (IP) twice a week for 6 or 10 weeks with or without ICM. Age-matched control mice were treated with a corn oil vehicle. For CCl₄+ICM treatment, mice were treated with CCl₄ for 2 weeks and then ICM (Department of Pharmaceutical Sciences at UConn) was dissolved in PEG (Sigma) and treated with mice (10 mg/kg) every day for 4 or 8 weeks in combination with CCl₄. To induced overexpression of HMGB2 in liver, AAV8-Hmgb2 or a control AAV8-GFP was given to mice by tail vein injection at a dose of 5×10¹⁰ GC. To knockdown HMGB2 in mouse livers, AAV-shHmgb2 or a control AAV-shGFP was delivered to mice at a dose of 2×10¹⁰ GC by tail vein injection. One week later, mice injected with AAVs were treated with CCl₄ or CCl₄+ICM as described above and sacrificed. Livers were removed and snap-frozen in liquid nitrogen for RNA and protein analysis. For the collection of mouse fetal livers, pregnant female mice were sacrificed at E12.5, E15, and E18.5 and livers were removed from the embryos. Additionally, post-natal mouse livers were harvested at postnatal day P1 and P60.

### Example 3

### Hepatic Overexpression of HMGB2 Augments CCl4-Incuded Liver Fibrosis

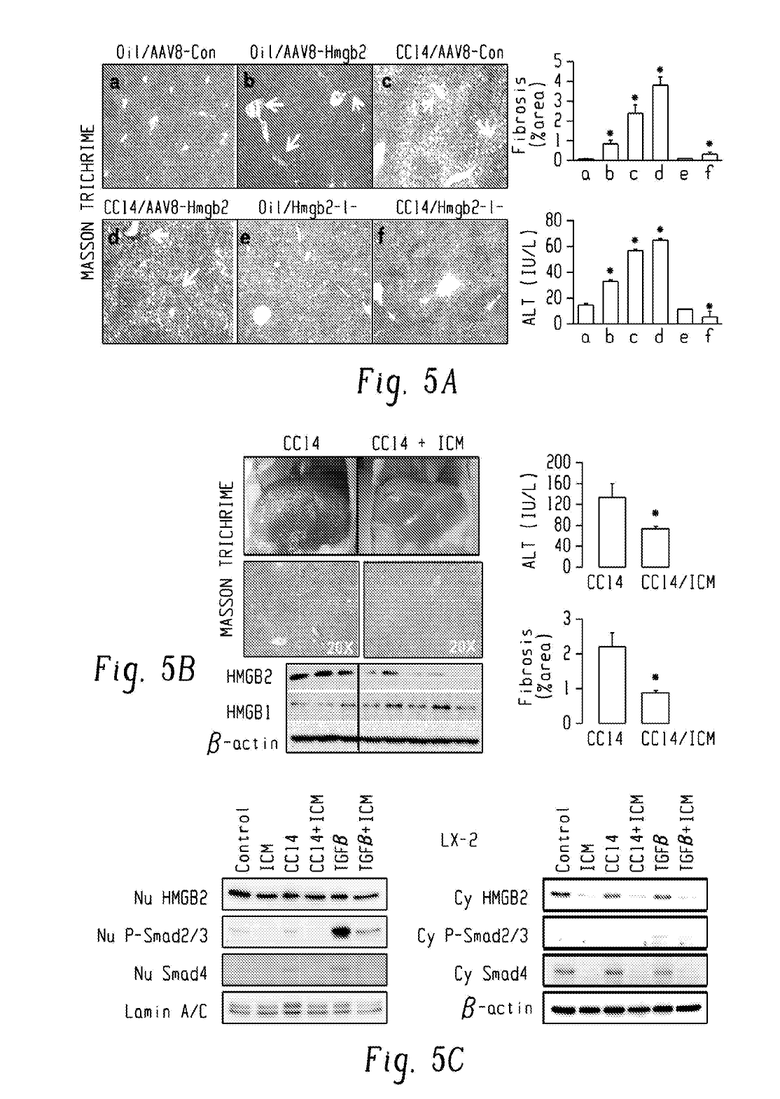

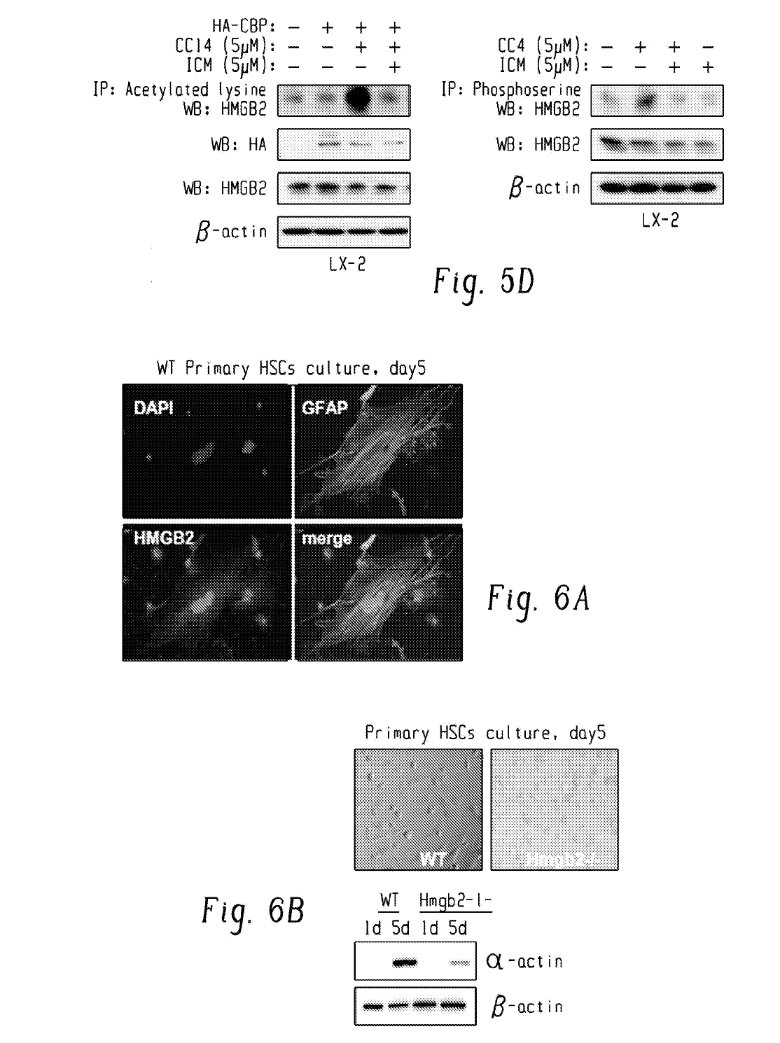

Adeno-associated virus AAV8 mediated gene delivery showed a long-lasting effect with no inflammation as compared to adenovirus. Therefore we generated AAV8-Flag-HMGB2 using a specific promoter thyroxine binding globulin (TBG) for hepatic directed delivery of the HMGB2 gene. A well-established liver fibrosis model induced by carbon tetrachloride (CCl₄) (850 μl/kg ip, twice a wk for 6 wks) was used to examine the effect of HMGB2 in combination with CCl4. One week after administration of AAV8-HMGB2 via the tail vein, WT mice (C57BL/6) were injected with CCl₄ or corn oil. Animals were sacrificed to harvest tissues 72 hr after the last injection. Overexpression of HMGB2 initiates liver fibrosis and enhances CCl₄-induced liver fibrosis, whereas HMGB2-deficiency (HMGB2−/−) prevents liver fibrosis and serum ALT levels (FIG. 5A). FIG. 5B shows that ICM decreases HMGB2 protein, liver fibrosis and serum ALT. In LX2 cells, ICM decreases Smad signaling and reduces cytoplasm HMGB2 protein (FIG. 5C). ICM decreases HMGB2 acetylation by CBP/CCl₄ and phosphorylation by CCl₄ (FIG. 5D).

### Example 4

### HMGB2−/− Primary Stellate Cells have Delayed Differentiation and Alpha-SMA Activation

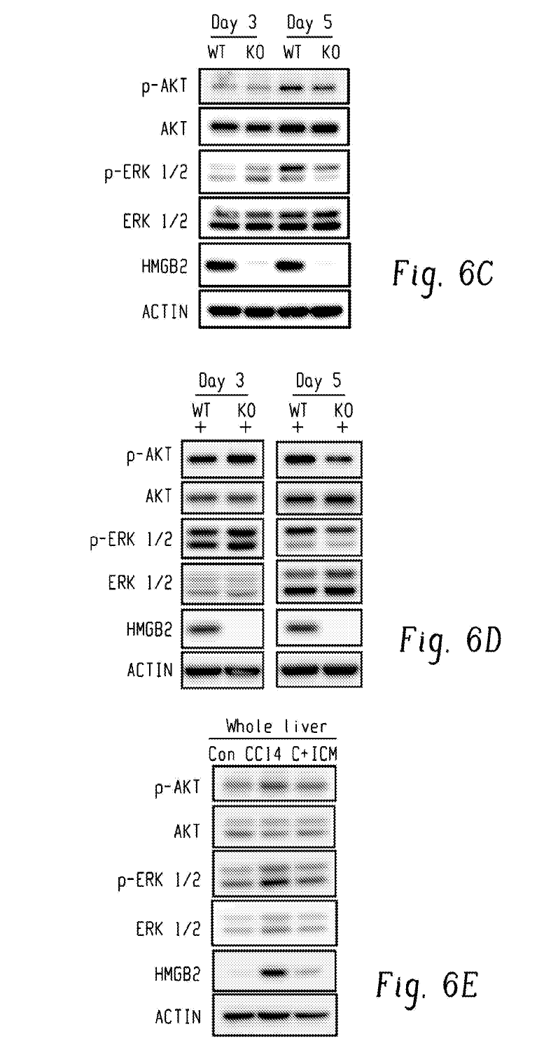

FIG. 6 shows that HMGB2−/− primary stellate cells have delayed differentiation (FIG. 6A) and alpha-SMA activation (FIG. 6B). The activation of pAKT and pERK1/2 is diminished in HMGB2−/− stellate cells cultured in vitro (FIG. 6C). HMGB2−/− primary stellate cells show diminished response to PDGF-induced pAKT and pERK1/2 activation (FIG. 6D). ICM reduces pAKR and pERk1/2 activation and HMGB2 protein (FIG. 6E). CCl₄ significantly induces HMGB2 protein which is blocked by ICM (FIG. 6F). The induction of HMGB2 protein by CCl₄ is gone after stopping CCl₄ treatment or by ICM.

Primary Mouse Hepatic Stellate Cells (HSCs) isolation and in vitro activation: Primary mouse HSCs were isolated from 8 to 12 weeks old wild type or Hmgb2^{-/-} mice, as described previously with modifications. Briefly, mice were anesthetized with ketamine/xylazine mixture. The liver was perfused with Hank's balanced salt solution (HBSS) supplemented with 0.5 mM EGTA through the cannulated portal vein, followed by perfusion with HBSS supplemented with 0.5 mg/ml pronase (Sigma) and HBSS with 0.14 mg/ml collagenase (Sigma). The liver was dissected out from the mouse and dissociated cells were dispersed gently in HBSS. Primary HSC was isolated by three-layer discontinuous density gradient centrifugation with 25% and 50% percoll (Sigma) and plated on non-coated culture dishes in DMEM supplemented with 10% FBS, 100 IU/ml penicillin, and 100 □g/ml streptomycin. For culture-activated HSC studies, the medium of primary HSCs was replaced every day up to 7 days. For PDGF treatment, primary HSC cells were serum starved for 24 hr and then treated with PDFG for 3 or 5 days.

### Example 5

### HMGB2 Protein is Highly Induced in Liver Progenitor Cell TIC Cells

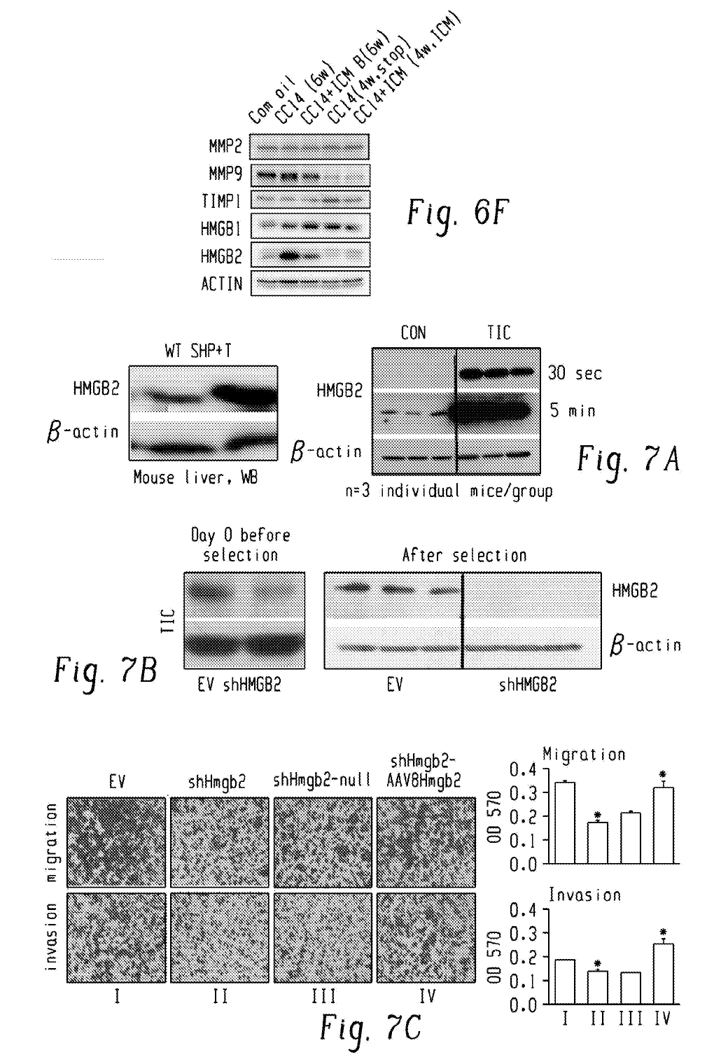

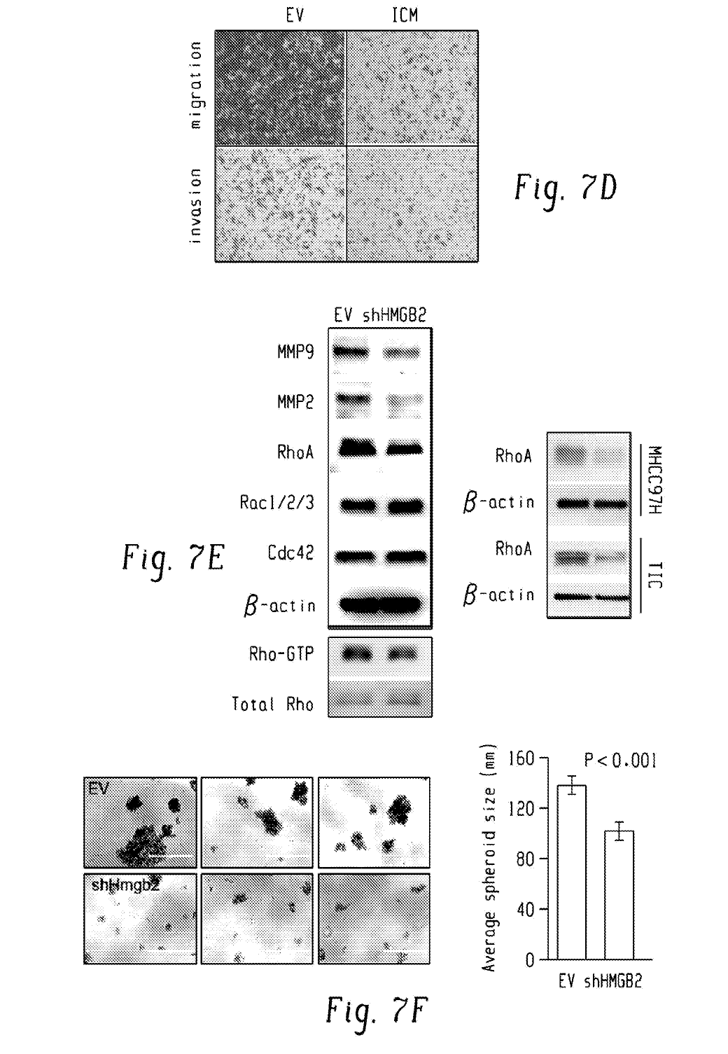

FIG. 7 shows that HMGB2 protein is highly induced in liver progenitor cell TIC cells (FIG. 7A). Generating HMGB2 stable knockdown TIC cells (FIG. 7B). shHMGB2 cells show decreased cell migration and invasion, which are reversed by HMGB2 overexpression (FIG. 7C). ICM markedly blocks TIC cell migration and invasion (Figure D). shHMGB2 or ICM treated cells show decreased MMP and Rho signaling (FIG. 7E). shHMGB2 cells show decreased spheroid size (FIG. 7F).

### Example 6

### Mouse Serum HMGB2 protein Levels are increased in 10 Wks CCl₄ Treated Mice

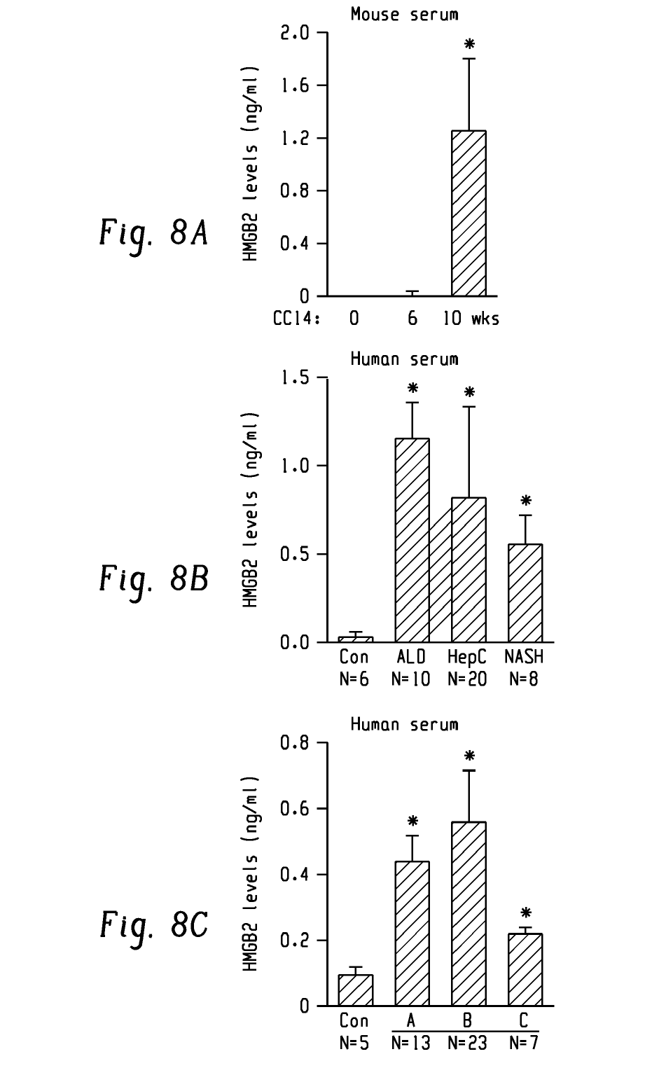

FIGS. 8A-C show that mouse serum HMGB2 protein levels are increased in 10 wks CCl4 treated mice (more severe stage of liver fibrosis) (FIG. 8A). In human alcoholic liver cirrhosis (ALD), HepC and NASH cirrhosis, HMGB2 protein levels are markedly increased (FIG. 8B-FIG. 8C). Thus, HMGB2 may serve as a new biomarker to indicate the stage of liver fibrosis/cirrhosis.

The levels of HMGB2 in serum samples from mice treated with CCl₄ and from patients who were associated with liver diseases were determined using ELISA kits (LifeSpan BioSciences) according to the manufacturer's instructions.

### Example 7

### High HMGB2 Expression is Correlated with Human NASH Cirrhosis and Advanced Fibrosis

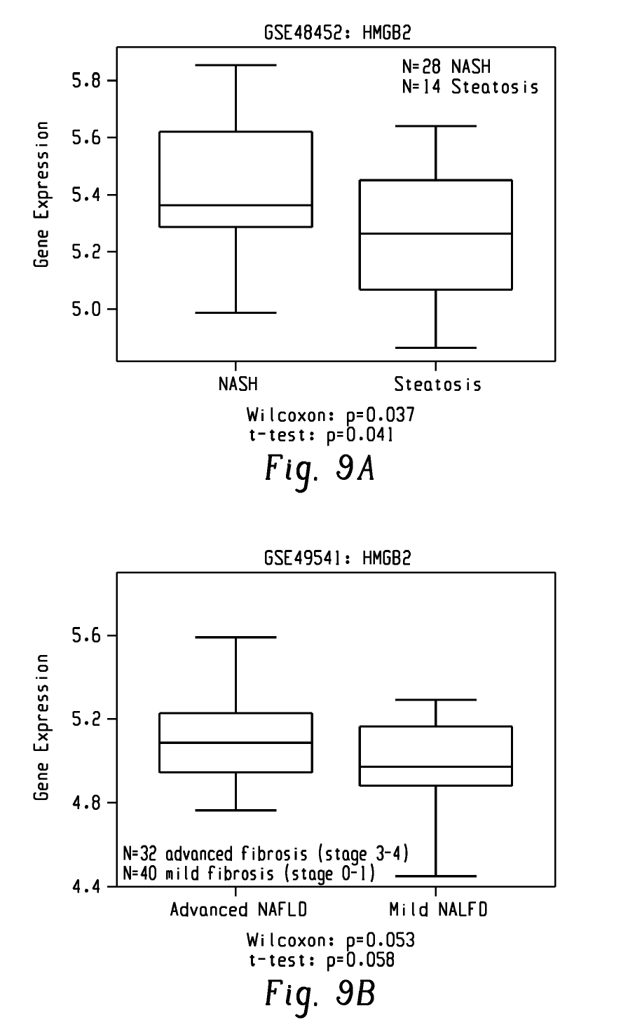

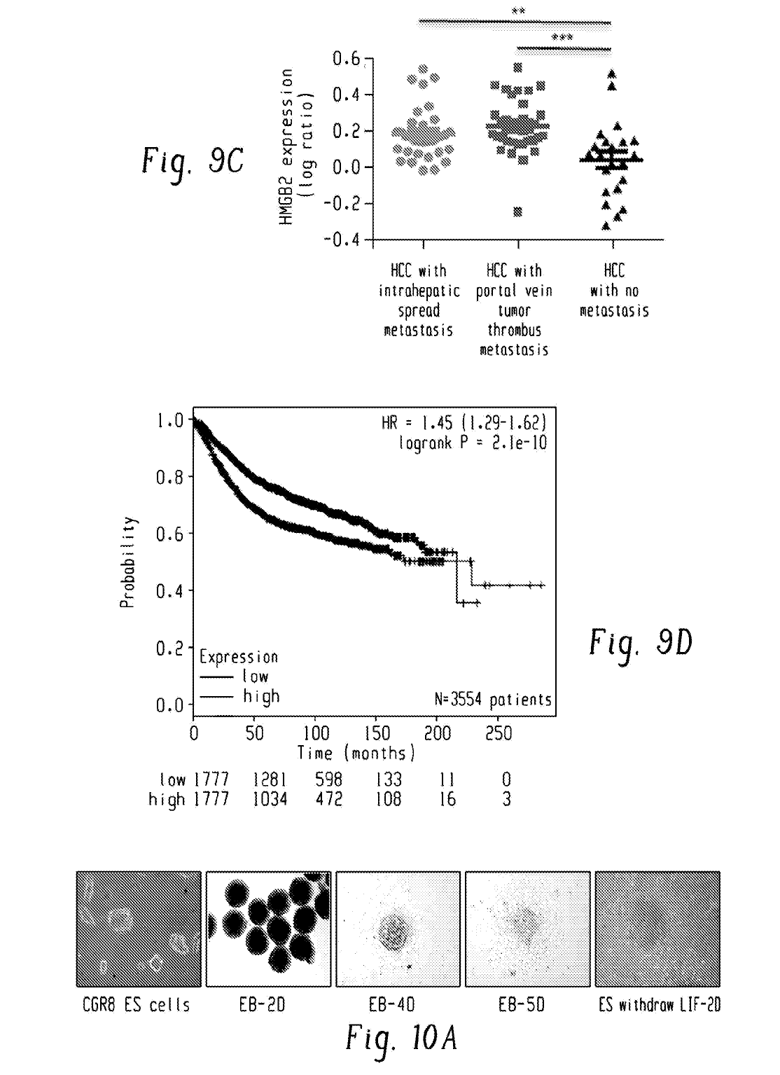

FIG. 9 shows that High HMGB2 expression is correlated with human NASH cirrhosis (FIG. 9A) and advanced fibrosis (FIG. 9B). HMGB2 expression is upregulated in human metastasis HCC (FIG. 9C) and its high expression is correlated with poor patient survival (FIG. 9D).

### Example 8

### HMGB2 is Positively Associated with OCT4 and SOX2 During EB Formation

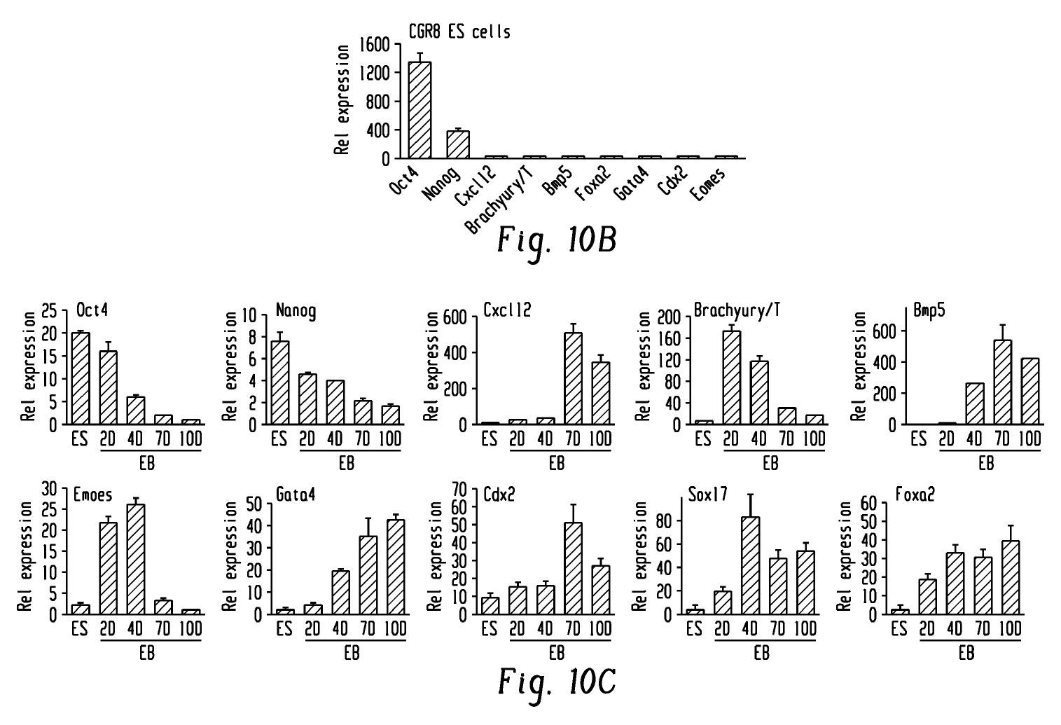

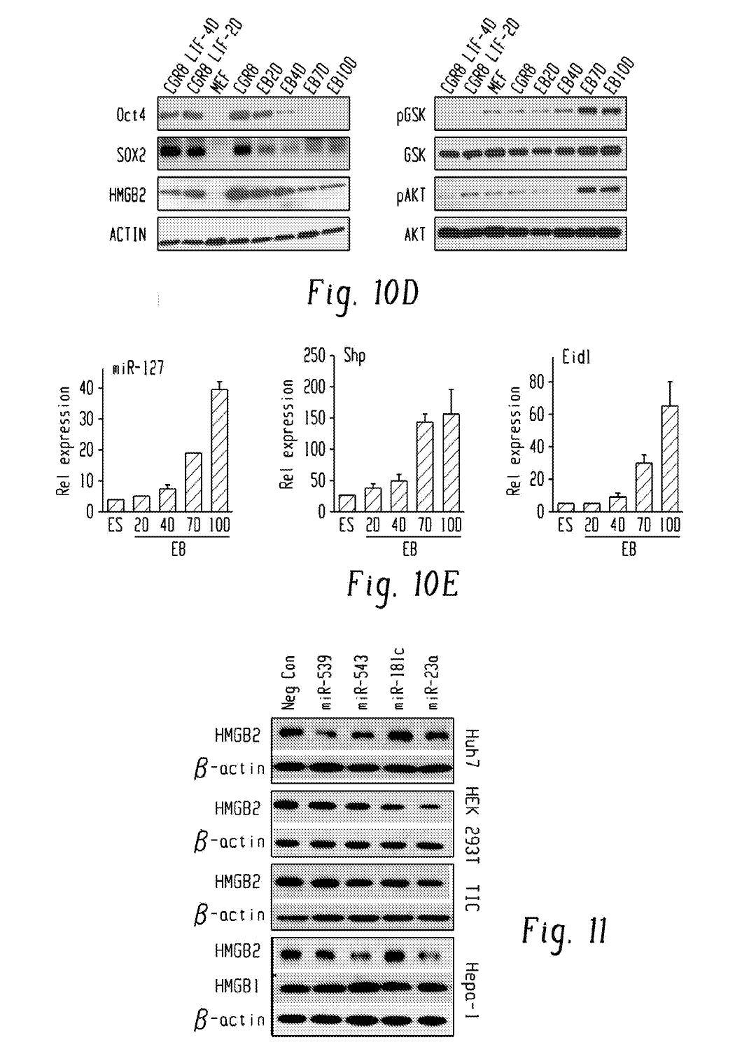

FIGS. 10A-C show the establishment of CGR8 cell culture and differentiation. FIG. 10A shows microscopic image of the mouse ES cells and EB outgrowths. Undifferentiated feeder-free CGR8 ES cells were maintained on 0.1% gelatin coated 6-cm tissue-culture dish in ES medium containing 10 ng/ml Leukemia inhibitory factor (LIF). For EB formation, CGR8 ES cells were trypsinized into single cells and cultured in ES medium devoid of LIF in hanging drops (900 cells per EB) for 2 d and in suspension culture for a further 2 d. Then, EBs were transferred onto gelatin coated tissue-culture plate for further spontaneously differentiation. ES withdraw LIF 2d, CGR8 ES cells were cultured in ES medium devoid of LIF for 2 d.

FIG. 10B shows qPCR of ES markers and three-germ layer genes mRNA in undifferentiated CGR8 ES cells. FIG. 10C shows qPCR of ES markers and three-germ layer genes mRNA during ES cell differentiation. FIG. 10D shows Western Blot of ES markers and HMGB2 during ES cell differentiation. FIG. 10D shows qPCR of miR-127, Shp and Eidl mRNA during ES cell differentiation.

HMGB2 is high in undifferentiated CGR8 but its levels diminish during differentiation along with OCT4 and SOX2 (FIG. 10D). miR-127 and SHP mRNA is increased during differentiation (FIG. 10E). Conclusions: The decreased HMGB2 expression is required for CGR8 cell differentiation. miR-127 and SHP mRNA is reversely correlated with HMGB2 expression, suggesting a negative regulation.

### Example 9

### Inhibition of HMGB2 Protein By miRNAs in Both Human and Mouse Cells

We identified conserved binding sites for miR-539, miR-543, miR-181c, and miR-23α in human and mouse HMGB2 3′UTRs (not sho4wn). We tested the inhibition of HMGB2 protein by these miRNAs in both human and mouse cells. We tested the inhibition of HMGB2 protein by these miRNAs in both human and mouse cells. Overexpression of miR-539, miR-543 and miR-23a decreased HMGB2 protein in Huh7, HEK293T, Hepa1, or TIC (FIG. 11), whereas miR-539 and miR-181c inhibited HMGB2 protein only in Huh7 and HEK293T, respectively. The high basal level of HMGB2 protein in HCC cells was in agreement with its induction in fibrotic/cirrhotic liver; the latter often progresses to HCC. The results demonstrate that HMGB2 protein undergoes miRNA mediated translational repression.

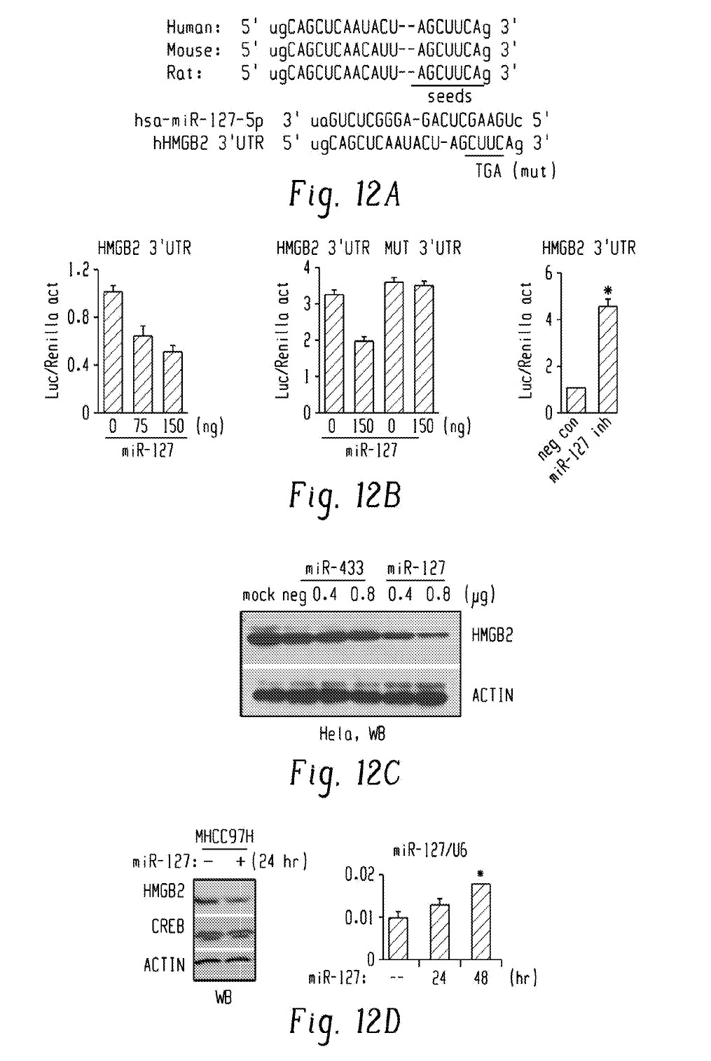

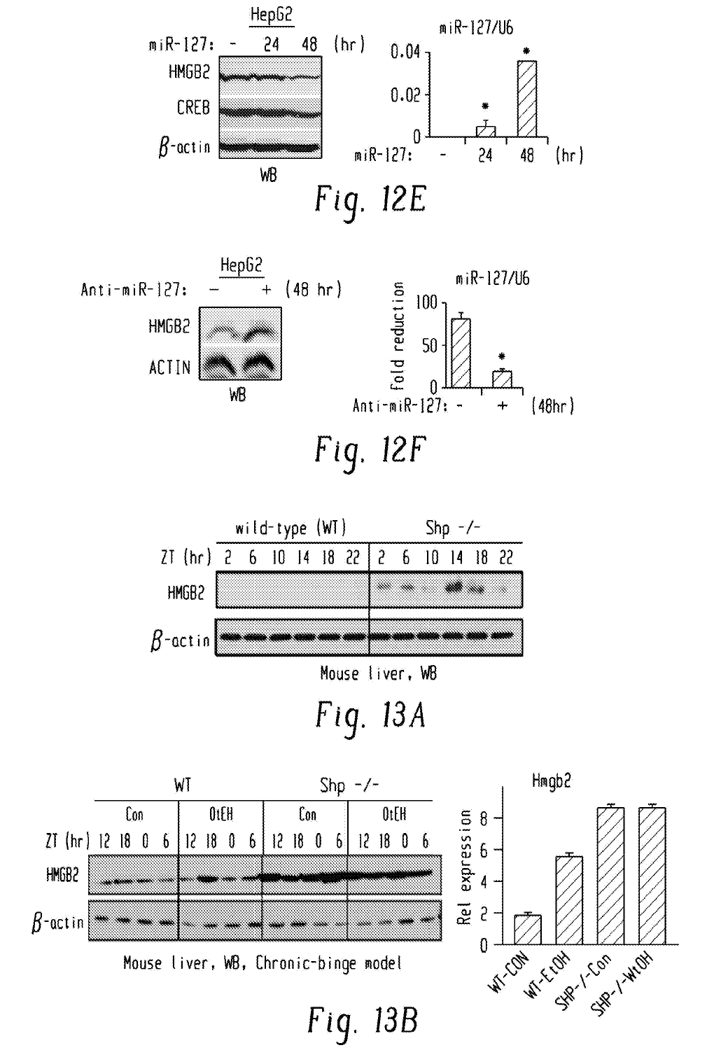

FIGS. 12A-F show that miR-127 targets the HMGB2 3′UTR (FIG. 12A-FIG. 12B) and decreases HMGB2 protein in HCC cells (FIGS. 12C-F). FIG. 12A is a sequence alignment of miR-127 and the 3′-UTR of HMGB2 in human, mouse and rat. The seed match region is indicated by underline. mut, mutant FIG. 12B is a Luciferase reporter assay. Left, HEK293T cells were transfected with HMGB2 3′UTR luciferase reporter with 0, 75 or 150 ng miR-127; Middle, HEK293T cells were transfected with wild-type or mutant HMGB2 3′UTR with or without 150 ng miR-127. Right, HEK293T cells were transfected with HMGB2 3′UTR with or without miR-127 antagomir. Luciferase activity was normalized to Renilla activity. In FIG. 12C, Western blotting was performed to determine endogenous HMGB2 protein in Hela cells that were transfected with pTarget, pTarget-miR-433, or pTarget-miR-127. β-Actin served as an internal control. In FIG. 12D, Left, western blot was performed to determine HMGB2 and CREB protein level in MHCC97H cells transfected with miR-127 for 24 hrs. Right, qPCR analysis of miR-127 level in MHCC97 cells transfected with miR-127 for 0, 24, 48 hrs. In FIG. 12E, Left, western blot was performed to determine HMGB2 and CREB protein level in HepG2 cells transfected with miR-127 for 0, 24, 48 hrs. Right, qPCR analysis of miR-127 level in HepG2 cells transfected with miR-127 for 0, 24, 48 hrs. In FIG. 12F, Left, western blot was performed to determine HMGB2 protein level in HepG2 cells transfected with or without miR-127 antagomir (anti-mir-127) for 48 hrs. Right, qPCR analysis of miR-127 level in HepG2 cells transfected with anti-miR-127 for 48 hrs.

HMGB2 protein is negatively regulated by miR-127. Thus HMGB2 is a downstream target of miR-127.

### Example 10

### HMGB2 is Regulated by E2F1/SHP/EID1 Network

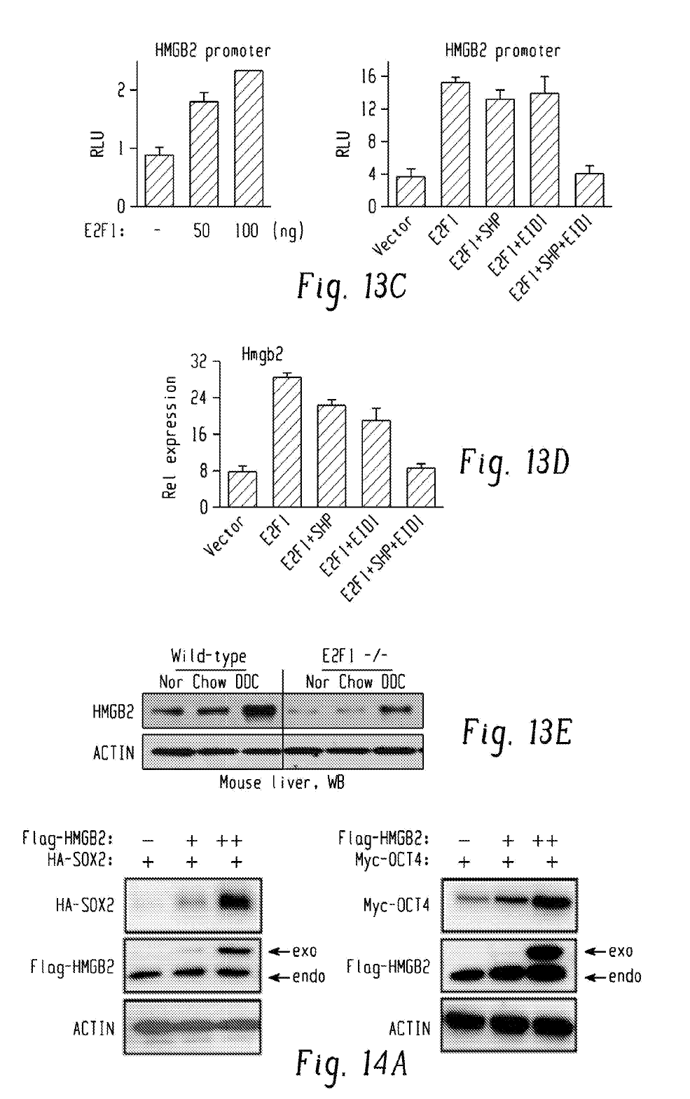

SHP functions as a transcriptional repressor in the nuclear receptor superfamily. We recently elucidated SHP in liver circadian clock mediated lipid and metabolic homeostasis. FIG. 13A is a Western blot of HMGB2 protein in wild-type or Shp-deficiency mouse liver An increased rhythmic expression of HMGB2 protein (FIG. 13A) and mRNA (not shown) was seen in SHP−/− vs WT liver (pooled equal amount of protein from 5 mice/group). The results suggest a transcriptional repression of HMGB2 by SHP. FIG. 13A is a Western blot (Left) and qPCR (Right) of HMGB2 in wild-type or Shp-deficiency mouse liver treated with or without EtOH. HMGB2 protein is also upregulated in alcohol-fed SHP−/− mouse liver (FIG. 13B). FIG. 13C is a Luciferase reporter assay. Left, HEK293T cells were transfected with HMGB2 promoter reporter with 0, 50 or 100 ng E2F1 expression vector. Right, HEK293T cells were transfected with HMGB2 promoter reporter with E2F1, SHP and EID expression plasmid alone or in combination. Luciferase activity was normalized to Renilla activity. FIG. 13D shows qPCR of HMGB2 mRNA in HEK293T cells that transfected with E2F1, SHP and EID1 expression vectors alone or in combination. HMGB2 promoter (FIG. 13C) and mRNA (FIG. 13D) are activated by E2F1 transcription factor, which are inhibited by SHP (FIG. 13C). FIG. 13E is a Western blot of HMGB2 in wild-type or E2f1-deficiency mouse liver fed with nor, Chow or DDC diet. HMGB2 protein is diminished in E2F1−/− mouse liver (FIG. 13E).

E2F1 functions as a transcriptional activator of HMGB2 gene transcription. SHP functions as a transcriptional repressor of HMGB2 gene transcription by inhibiting E2F1 activity.

### Example 11

### HMGB2 Modulates ES Pluripotency Via Stabilizing OCT4 and SOX2

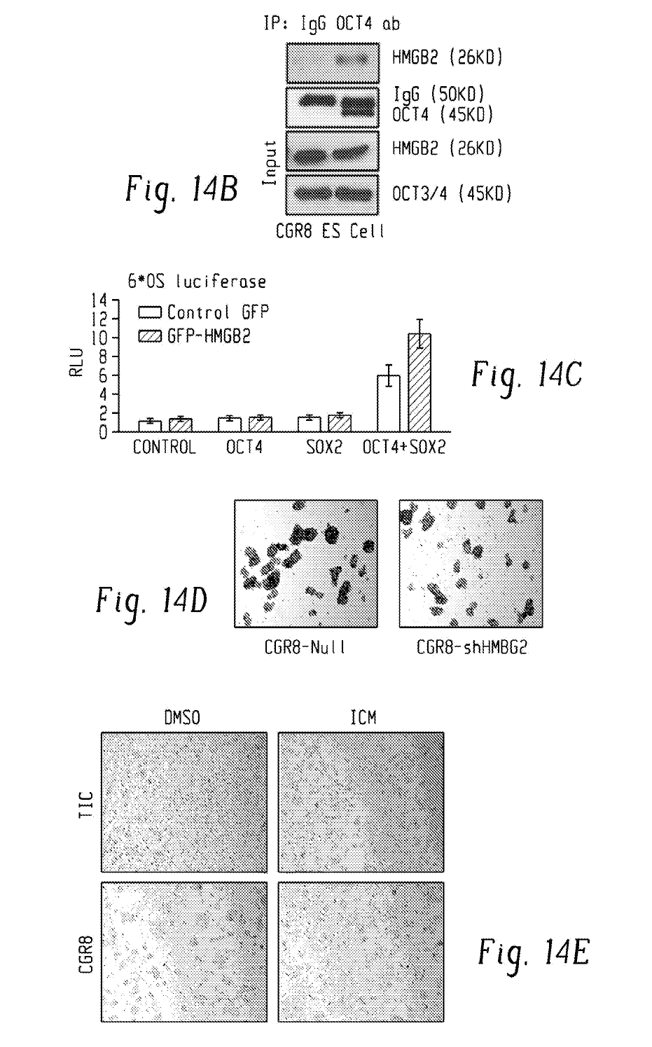

FIG. 14 shows that overexpression of HMGB2 increases SOX2 and OCT4 protein levels (FIG. 14A), interacts with OCT4 protein (FIG. 14B), and enhances OCT4/SOX2 transcriptional activity (FIG. 14C). FIG. 14A is Left, Western blot of HMGB2 and SOX2 protein in HEK293T cells transfected with 0.5 μg HA-50X2 expression plasmid and 0, 1 or 2 μg Flag-HMGB2 expression plasmid. Right, Western blot of HMGB2 and OCT4 protein in HEK293T cells transfected with 0.5 μg HA-OCT4 expression plasmid and 0, 1 or 2 μg Flag-HMGB2 expression plasmid. FIG. 14B shows endogenous co-immunoprecipitation. CGR8 cells were lysed, co-immunoprecipitated with OCT4 antibody, followed by Western blot with HMGB2 or OCT4 antibodies. FIG. 14 C is a luciferase reporter assay. HepG2 cells were infected with AAV8-GFP or AAV8-HMGB2 24 hrs before being transfected with 6*OS luciferase reporter and OCT4, SOX2 expression plasmids alone or in combination. FIG. 14D shows microphage of EB formation from CGR8 cells infected with AAV8-Null or AAV8-shHMGB. HMGB2 knockdown decreases spheroid formation of CGR8 cells (FIG. 14D). FIG. 14D shows microphage of TIC cells (Upper) or CGR8 cells (Bottom) treated with 5 μM HMGB2 inhibitor Inflachromene (ICM) or DMSO. ICM decreases TIC cell proliferation and induces CGR8 differentiation (FIG. 14E).

HMGB2 increases protein stability of SOX2 and OCT4, interacts with OCT4, enhances SOX2 and OCT4 transcriptional activity. Knockdown HMGB2 by shRNA in CGR8 decreases spheroid size. Knockdown HMGB2 by ICM in TIC cells decrease cell proliferation. Knockdown HMGB2 by ICM in CGR8 promotes differentiation.

### Example 12

### Effects of miR-127 Overexpression

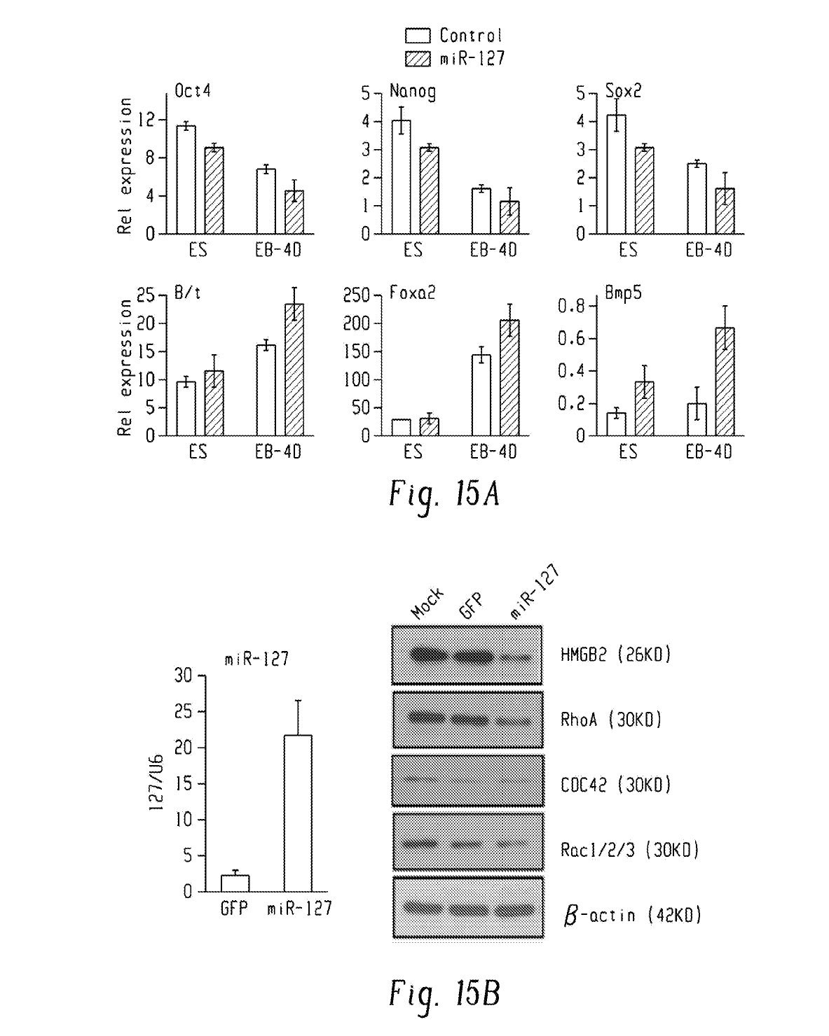

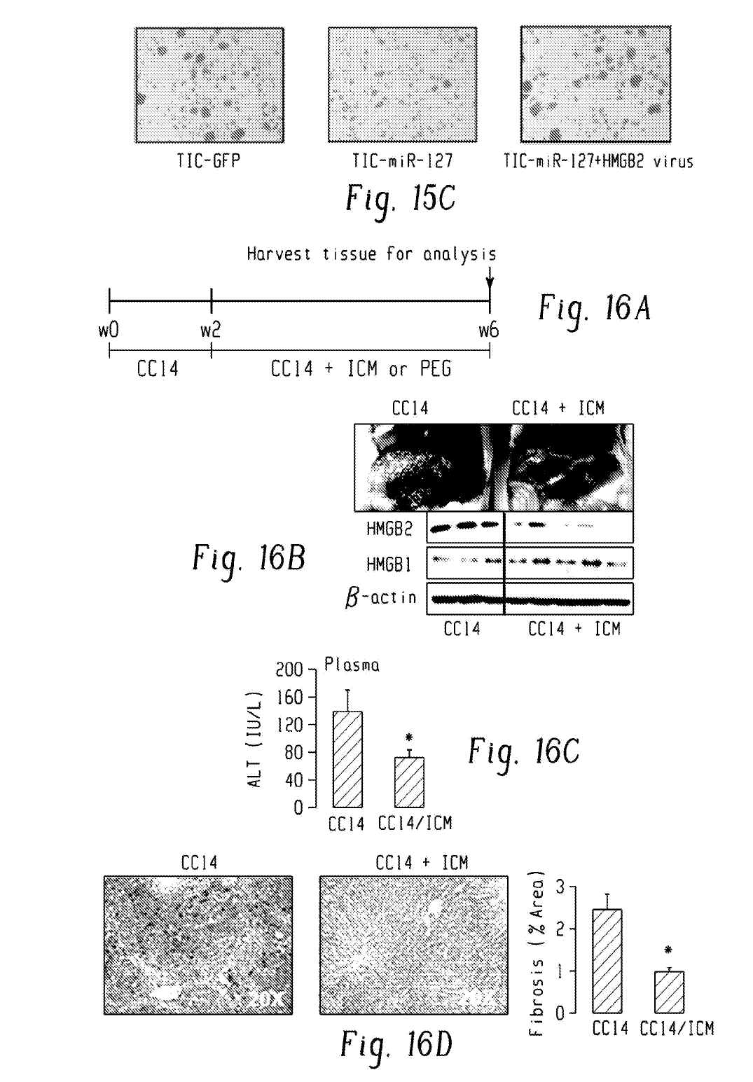

FIG. 15A shows qPCR of ES markers and three-germ layer genes mRNA in undifferentiated CGR8 ES cells and 4d EBs which was infected with miR-127 or GFP lentivirus. FIG. 15B shows Left, qPCR analysis of miR-127 level in TIC cells which were infected with miR-127 lentivirus. Right, western blot of HMGB2, RhoA, CDC42, Rac1/2/3 in TIC cells which were infected with miR-127 or GFP lentivirus. FIG. 15C shows a Spheroid assay of TIC cells infected with miR-127, GFP or miR-127 combined with HMGB2 lentivirus.

miR-127 overexpression decreases stem cell marker (Oct4, Nanog, Sox2) expression and increases differentiation markers (Bmp5, Foxa2). miR-127 downregulates HMGB2 protein and Rhos/CDC42/Rac signaling, and spheroid formation.

Summary: HMGB2 promotes stellate cell activation and hepatic inflammation and HMGB2-deficiency by gene knockout or ICM prevents liver fibrosis progression by diminishing TGFbeta/Smad/AKT/ERK1/2 signaling.

HMGB2 promotes liver progenitor TIC cell migration/invasion. ICM or shHMGB2 blocks it by downregulating MMP/RhoA/Rac signaling.

HMGB2 protein is secreted in human liver fibrosis/cirrhosis and its expression is associated with disease severity and patient survival.

HMGB2 is highly expressed in CGR8 stem cell. HMGB2 interacts with SOX2/OCT4 to enhance their transcriptional activity.

HMGB2 protein is inhibited by miR-127 and its gene transcription is inhibited by SHP involving E2F1/EID1.

Knockdown of HMGB2 by shHMGB2, ICM or miR-127 promotes CGR8 differentiation.

### Example 13

### Therapeutic Efficacy of Selective Small Molecular Inhibitors of HMGB2

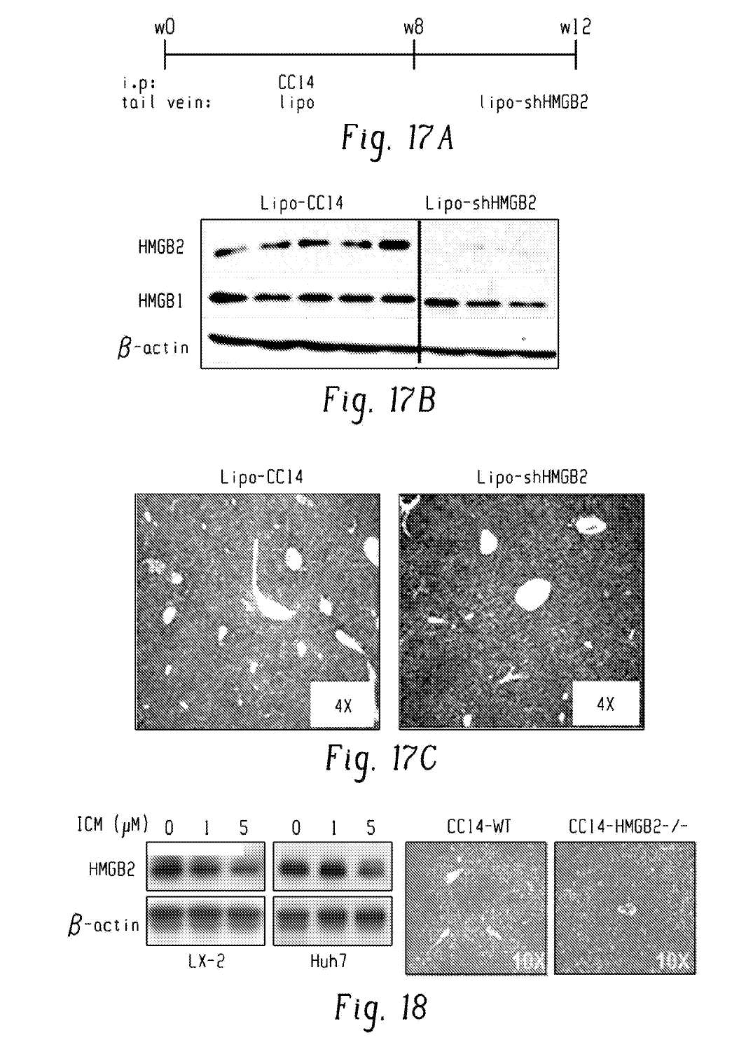

To test the therapeutic efficacy of selective small molecular inhibitors of HMGB2, a small molecule inhibitor of HMGB2, inflachromene (ICM), was synthesized. The first group of mice received CCl₄ to induce liver fibrosis and the second group received CCl₄+ICM (FIG. 16A). Intriguingly, ICM treated mice showed improved liver gross morphology and a reduction in liver HMGB2 protein induced by CCl₄ (FIG. 16B). This was accompanied by the decreased plasma ALT levels (FIG. 16C) and diminished liver fibrosis (FIG. 16D). We tested the effect of shHMGB2. We used CCl₄ to induce liver fibrosis for 8 wks and then introduced liposome-shHMGB2 to knockdown the endogenous HMGB2 induced by CCl₄ (FIG. 17A-FIG. 17B). We observed a marked reduction in liver fibrosis by lipo-shHMGB2 (FIG. 17C). A third group of mice received CCl₄ for 12 wks and lipo-shHMGB2 for the last 4 wks. Under this condition, HMGB2 was constantly induced by CCl₄, thus efficient knockdown by lipo-shHMGB2 was not achieved (not shown). Overall, the results suggest that diminishing HMGB2 expression may serve as a promising therapeutic strategy to prevent or reverse liver fibrosis.

ICM was described as a dual inhibitor of both HMGB1 and HMGB2 in a neuroinflammation model. Interestingly, ICM appeared to be more selective to downregulate HMGB2 protein with less effect on HMGB1 protein in mouse liver (FIG. 16), suggesting that the action of ICM may be tissue or cell type specific. In addition, ICM dose-dependently decreased HMGB2 protein in LX-2 and Huh7 cells (FIG. 18 left) (Note: HMGB1 protein could not be detected under the same conditions). Moreover, CCl₄-induced liver fibrosis was diminished, although not abolished, in whole-body HMGB2−/− mice (FIG. 18 right). Our results not only provided a valuable pharmacological validation of HMBG2 as a potential therapeutic target in the liver but also provided an outstanding departure point for the development of superior agents with greater potency, a more restricted pharmacological profile and excellent physicochemical properties. Selectivity is viewed as a desirable property in order to avoid unexpected and undesirable side effects that could be produced through inhibition of HMGB1 in other organs/tissues with non-selective inhibitors.

### Example 14

### ICM Analogs

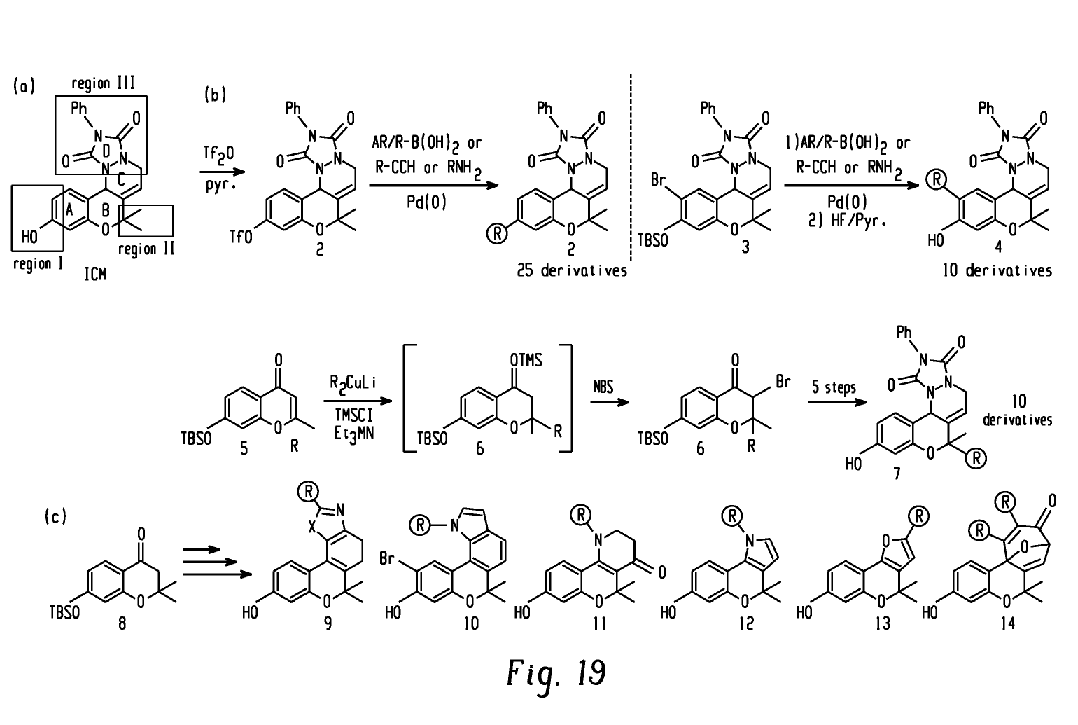

An analysis of the initial lead structure suggests several domains for generating analogs including the region aryl ring of the benzopyranone core (especially in regard to the phenolic hydroxyl), the flanking groups of the pyran ether oxygen in region II and the potential for substantial modification in the fused region III domain. The analog campaign will treat each region independently, holding the other two domains constant (FIG. 19). The routes utilized to the region I and region II analogs represent simple modification of the known synthesis where diverse aromatic rings x or adehydes/ketones x are utilized to generate a diverse range dienes x that can be reacted with the N-phenytriazolinedione dienonophile to yield approximately 25 derivatives at each region. From the minimal, published SAR on the initial library hit, we anticipate that there will be substantial freedom-to-operate in region III which offers the potential for “scaffold hopping” that may reveal more isoform selective chemotypes. For these derivatives, we will utilize the two isomeric dienes x and x we have already prepared at a branch point to 25 initial new scaffolds by exploiting the diversity potential of a conjugated diene. More promising scaffolds that emerge can be further tuned in focused compound arrays such as those illustrated for the region I and II derivatives. In brief, we will use a variety of heteroatom insertion, Diels-Alder reactions, and higher-order cylcloadditions to annulate 5,6 and 7-membered rings on to the benzopyran core.

Promising lead compounds (˜20) will be evaluated in both liver microsomes and mouse hepatocytes to determine half-life and to identify metabolites arising from both Phase I and Phase II transformations by LC/MS/MS and metabolite synthesis. This information can be used to redesign leads with improved pharmacokinetics by limiting specific metabolic liabilities as needed.

We will treat LX-2 and Huh7 cells with 10 selected compounds and determine total and modified forms (phosphorylation, acetylation, and ubiquitination) of HMGB2 protein. HMGB1 protein will also be examined (neg. control).

### Example 15

### Liposome Mediated Delivery System to Target HMGB2 in Liver Fibrosis

Anionic lipids, a naturally occurring component of eukaryotic cell membranes, have been exploited as a potential drug delivery vector in recent years due to cytotoxicity problems associated with cationic vectors. The optimized anionic lipoplexes composed of physiologically safe components (divalent cations, anionic and fusogenic lipids) have been developed and investigated for plasmid DNA and siRNA delivery, exhibiting silencing efficiency equivalent to cationic formulations with negligible cytotoxicity and significantly improved serum stability. Such liposomes can be used to deliver small molecules, shRNA or miRNA to stellate cells in mouse liver using liposomes.

Anionic liposomes with Vitamin A (as a stellate cell targeting ligand) were prepared using a continuous ethanol injection system. Particle size and surface charge were controlled to optimize uptake into the cells via endocytosis. The formulation includes DPPG (1,2-dipalmitoyl-sn-glycero-3-phospho-(1′-rac-glycerol)), cholesterol and a fusogenic helper lipid, DOPE (1,2-dioleoyl-sn-glycero-3-phosphoethanolamine). The fusogenic lipid facilitates endosomal escape of the intact bioactive (siRNA, mRNA and/or small molecule inhibitor of HMGB2). Briefly, the lipids, a small molecule inhibitor (if applicable), and Vitamin A were dissolved in ethanol and injected into an aqueous phase to form monodispersed liposomes using a coaxial turbulent jet in co-flow. Liposomes were then coated with a positive-charge species (e.g. Ca2+ or chitosan), followed by the addition of siRNA or miRNA. The combination of all these species results in the formation of lipoplexes (liposomes complexes) (FIG. 20).

The advantages of this system are: the gene therapeutic and/or small molecule inhibitor is protected from the in vivo environment and will be safely delivered to the target site; it is composed of anionic lipids (low cytotoxicty); and includes Vitamin A for targeting to the stellate cells. This system is used to deliver shRNA or miRNA, and/or co-deliver a small molecule inhibitor of HMGB2 in combination with siRNA or miRNA.

Using the optimized anionic formulation of lipoplexes, the in vivo efficacy of 3 promising compounds (target HMGB2 protein modification) identified in 3A, alone or in combination with shHMGB2 (target HMGB2 gene transcription) or miR-23α (target HMGB2 protein translation) will be determined. The CCl₄ model has been used to study the mechanism regulating the reversibility/resolution of liver fibrosis, thus the priority will be focused on the CCl4 model.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A method of treating a subject in need of treatment for liver fibrosis, the method comprising administering a therapeutically effective amount of an inhibitor of high mobility box 2 gene (HMGB2).

2. The method of claim 1, wherein the subject has been diagnosed with chronic liver injury; cirrhosis; chronic hepatitis B; chronic hepatitis C; nonalcoholic steatohepatitis (NASH); a parasitic liver infection; bacterial liver infection; primary or secondary liver cancer; a metabolic disorder that causes liver injury; alcoholic liver disease; Wilson's disease; nonalcoholic fatty liver disease; autoimmune liver disease; cholestatic liver disease; or biliary obstruction.

3. The method of claim 1, wherein the inhibitor of HMGB2 is a micro RNA or an interfering RNA.

4. The method of claim 3, wherein the inhibitor of HMGB2 is miR-127, miR-539, miR-543, miR-23a, or a small hairpin RNA.

5. The method of claim 4, wherein the small hairpin RNA is shRNA-HMGB2.

6. The method of claim 1, wherein the inhibitor of HMGB2 is an inflachromene analog of Formula I: 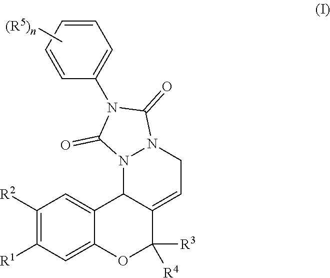
wherein
R¹ and R² are each independently hydrogen, —OH, C₁-C₆ alkyl, C₁-C₆ alkoxy, C₂-C₆ alkenyl, C₂-C₆ alkynyl, C₂-C₆ alkanoyl, C₂-C₆ alkoxycarbonyl, mono- or di-C₁-C₆ alkylcarboxamide, mono- or di-C₁-C₆ alkylamino, aryl, (aryl)C₁-C₆ alkyl, substituted or unsubstituted (aryl)carbonyl, C₃-C₈ cycloalkyl, halogen, C₁-C₆ haloalkyl, C₁-C₆ haloalkoxy, heteroaryl, (heteroaryl)C₁-C₆ alkyl, heterocycloalkyl, —COOH, —CN, —NH₂, —NO₂, (C₁-C₆ alkyl)amido, (C₂-C₆ alkenyl)amido, or (C₂-C₆ alkynyl)amido;
R³ and R⁴ are each independently hydrogen, —OH, C₁-C₆ alkyl, C₁-C₆ alkoxy, C₂-C₆ alkenyl, C₂-C₆ alkynyl, C₂-C₆ alkanoyl, C₂-C₆ alkoxycarbonyl, mono- or di-C₁-C₆ alkylcarboxamide, mono- or di-C₁-C₆ alkylamino, aryl, (aryl)C₁-C₆ alkyl, substituted or unsubstituted (aryl)carbonyl, C₃-C₈ cycloalkyl, halogen, C₁-C₆ haloalkyl, C₁-C₆ haloalkoxy, heteroaryl, (heteroaryl)C₁-C₆ alkyl, heterocycloalkyl, —COOH, —CN, —NH₂, —NO₂, (C₁-C₆ alkyl)amido, (C₂-C₆ alkenyl)amido, (C₂-C₆ alkynyl)amido, or R³ and R⁴ together can form a substituted or unsubstituted C₃-C₆ cycloalkyl, or substituted or unsubstituted C₃-C₆ heterocycloalkyl;
each instance of R⁵ is hydrogen, —OH, C₁-C₆ alkyl, C₁-C₆ alkoxy, C₂-C₆ alkenyl, C₂-C₆ alkynyl, C₂-C₆ alkanoyl, C₂-C₆ alkoxycarbonyl, mono- or di-C₁-C₆ alkylcarboxamide, mono- or di-C₁-C₆ alkylamino, aryl, (aryl)C₁-C₆ alkyl, substituted or unsubstituted (aryl)carbonyl, C₃-C₈ cycloalkyl, halogen, C₁-C₆ haloalkyl, C₁-C₆ haloalkoxy, heteroaryl, (heteroaryl)C₁-C₆ alkyl, heterocycloalkyl, —COOH, —CN, —NH₂, —NO₂, (C₁-C₆ alkyl)amido, (C₂-C₆ alkenyl)amido, or (C₂-C₆ alkynyl)amido; n is 0, 1, 2, or 3; or a pharmaceutically acceptable salt thereof; with the proviso that when R² and R¹ are both hydrogen then R¹, R³, and R⁴ are not —OH, methyl, and methyl, respectively.

7. The method of claim 6, wherein the inhibitor is inflamachromene.

8. The method of claim 1, wherein the inhibitor of HMGB2 comprises
an inflamochromene analog of Formula I, and
an inhibitory nucleic acid or a micro RNA 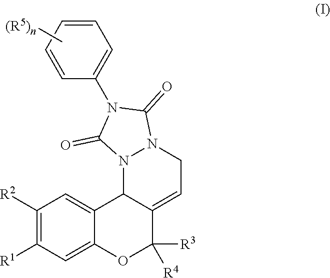
wherein
R¹ and R² are each independently hydrogen, —OH, C₁-C₆ alkyl, C₁-C₆ alkoxy, C₂-C₆ alkenyl, C₂-C₆ alkynyl, C₂-C₆ alkanoyl, C₂-C₆ alkoxycarbonyl, mono- or di-C₁-C₆ alkylcarboxamide, mono- or di-C₁-C₆ alkylamino, aryl, (aryl)C₁-C₆ alkyl, substituted or unsubstituted (aryl)carbonyl, C₃-C₈ cycloalkyl, halogen, C₁-C₆ haloalkyl, C₁-C₆ haloalkoxy, heteroaryl, (heteroaryl)C₁-C₆ alkyl, heterocycloalkyl, —COOH, —CN, —NH₂, —NO₂, (C₁-C₆ alkyl)amido, (C₂-C₆ alkenyl)amido, or (C₂-C₆ alkynyl)amido;
R³ and R⁴ are each independently hydrogen, —OH, C₁-C₆ alkyl, C₁-C₆ alkoxy, C₂-C₆ alkenyl, C₂-C₆ alkynyl, C₂-C₆ alkanoyl, C₂-C₆ alkoxycarbonyl, mono- or di-C₁-C₆ alkylcarboxamide, mono- or di-C₁-C₆ alkylamino, aryl, (aryl)C₁-C₆ alkyl, substituted or unsubstituted (aryl)carbonyl, C₃-C₈ cycloalkyl, halogen, C₁-C₆ haloalkyl, C₁-C₆ haloalkoxy, heteroaryl, (heteroaryl)C₁-C₆ alkyl, heterocycloalkyl, —COOH, —CN, —NH₂, —NO₂, (C₁-C₆ alkyl)amido, (C₂-C₆ alkenyl)amido, (C₂-C₆ alkynyl)amido, or R³ and R⁴ together can form a substituted or unsubstituted C₃-C₆ cycloalkyl, or substituted or unsubstituted C₃-C₆ heterocycloalkyl;
each instance of R⁵ is hydrogen, —OH, C₁-C₆ alkyl, C₁-C₆ alkoxy, C₂-C₆ alkenyl, C₂-C₆ alkynyl, C₂-C₆ alkanoyl, C₂-C₆ alkoxycarbonyl, mono- or di-C₁-C₆ alkylcarboxamide, mono- or di-C₁-C₆ alkylamino, aryl, (aryl)C₁-C₆ alkyl, substituted or unsubstituted (aryl)carbonyl, C₃-C₈ cycloalkyl, halogen, C₁-C₆ haloalkyl, C₁-C₆ haloalkoxy, heteroaryl, (heteroaryl)C₁-C₆ alkyl, heterocycloalkyl, —COOH, —CN, —NH₂, —NO₂, (C₁-C₆ alkyl)amido, (C₂-C₆ alkenyl)amido, or (C₂-C₆ alkynyl)amido; n is 0, 1, 2, or 3; or a pharmaceutically acceptable salt thereof; with the proviso that when R² and R⁵ are both hydrogen then R¹, R³, and R⁴ are not —OH, methyl, and methyl, respectively.

9. The method of any one or more of claims 1-8, wherein the inhibitor of HMGB2 is administered in the form of an anionic or cationic liposome particle.

10. The method of claim 9, wherein the anionic or cationic liposome particle comprises Vitamin A.

11. The method of claim 10, wherein the anionic liposome particle is coated with a positively-charged species.

12. A composition comprising an inflachromene analog of Formula I and a micro RNA or an interfering RNA that inhibits the expression of HMGB2, 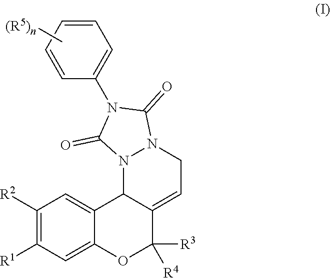
wherein
R¹ and R² are each independently hydrogen, —OH, C₁-C₆ alkyl, C₁-C₆ alkoxy, C₂-C₆ alkenyl, C₂-C₆ alkynyl, C₂-C₆ alkanoyl, C₂-C₆ alkoxycarbonyl, mono- or di-C₁-C₆ alkylcarboxamide, mono- or di-C₁-C₆ alkylamino, aryl, (aryl)C₁-C₆ alkyl, substituted or unsubstituted (aryl)carbonyl, C₃-C₈ cycloalkyl, halogen, C₁-C₆ haloalkyl, C₁-C₆ haloalkoxy, heteroaryl, (heteroaryl)C₁-C₆ alkyl, heterocycloalkyl, —COOH, —CN, —NH₂, —NO₂, (C₁-C₆ alkyl)amido, (C₂-C₆ alkenyl)amido, or (C₂-C₆ alkynyl)amido;
R³ and R⁴ are each independently hydrogen, —OH, C₁-C₆ alkyl, C₁-C₆ alkoxy, C₂-C₆ alkenyl, C₂-C₆ alkynyl, C₂-C₆ alkanoyl, C₂-C₆ alkoxycarbonyl, mono- or di-C₁-C₆ alkylcarboxamide, mono- or di-C₁-C₆ alkylamino, aryl, (aryl)C₁-C₆ alkyl, substituted or unsubstituted (aryl)carbonyl, C₃-C₈ cycloalkyl, halogen, C₁-C₆ haloalkyl, C₁-C₆ haloalkoxy, heteroaryl, (heteroaryl)C₁-C₆ alkyl, heterocycloalkyl, —COOH, —CN, —NH₂, —NO₂, (C₁-C₆ alkyl)amido, (C₂-C₆ alkenyl)amido, (C₂-C₆ alkynyl)amido, or R³ and R⁴ together can form a substituted or unsubstituted C₃-C₆ cycloalkyl, or substituted or unsubstituted C₃-C₆ heterocycloalkyl;
each instance of R⁵ is hydrogen, —OH, C₁-C₆ alkyl, C₁-C₆ alkoxy, C₂-C₆ alkenyl, C₂-C₆ alkynyl, C₂-C₆ alkanoyl, C₂-C₆ alkoxycarbonyl, mono- or di-C₁-C₆ alkylcarboxamide, mono- or di-C₁-C₆ alkylamino, aryl, (aryl)C₁-C₆ alkyl, substituted or unsubstituted (aryl)carbonyl, C₃-C₈ cycloalkyl, halogen, C₁-C₆ haloalkyl, C₁-C₆ haloalkoxy, heteroaryl, (heteroaryl)C₁-C₆ alkyl, heterocycloalkyl, —COOH, —CN, —NH₂, —NO₂, (C₁-C₆ alkyl)amido, (C₂-C₆ alkenyl)amido, or (C₂-C₆ alkynyl)amido; n is 0, 1, 2, or 3; or a pharmaceutically acceptable salt thereof; with the proviso that when R² and R¹ are both hydrogen then R¹, R³, and R⁴ are not —OH, methyl, and methyl, respectively.

13. The composition of claim 12, wherein the composition is in the form of an anionic or cationic liposome particle.

14. The composition of claim 13, wherein the anionic or cationic liposome particle comprises Vitamin A.

15. The composition of claim 14, wherein the anionic liposome particle is coated with a positively-charged species.

16. The composition of claim 12, wherein the micro RNA is miR-127, miR-539, miR-543, or miR-23a.

17. The composition of claim 12, wherein the interfering RNA is a small hairpin RNA.

18. A composition comprising an anionic or cationic liposome particle, comprising
Vitamin A, and
an inhibitor of HMGB2.

19. The composition of claim 18, wherein the inhibitor of HMGB2 is an inflamochromene analog of Formula I, or a micro RNA or an interfering RNA that inhibits the expression of HMGB2 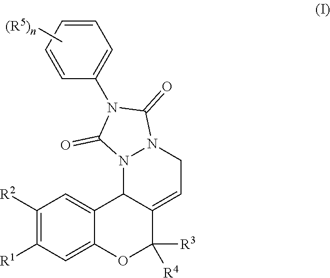
wherein
R¹ and R² are each independently hydrogen, —OH, C₁-C₆ alkyl, C₁-C₆ alkoxy, C₂-C₆ alkenyl, C₂-C₆ alkynyl, C₂-C₆ alkanoyl, C₂-C₆ alkoxycarbonyl, mono- or di-C₁-C₆ alkylcarboxamide, mono- or di-C₁-C₆ alkylamino, aryl, (aryl)C₁-C₆ alkyl, substituted or unsubstituted (aryl)carbonyl, C₃-C₈ cycloalkyl, halogen, C₁-C₆ haloalkyl, C₁-C₆ haloalkoxy, heteroaryl, (heteroaryl)C₁-C₆ alkyl, heterocycloalkyl, —COOH, —CN, —NH₂, —NO₂, (C₁-C₆ alkyl)amido, (C₂-C₆ alkenyl)amido, or (C₂-C₆ alkynyl)amido;
R³ and R⁴ are each independently hydrogen, —OH, C₁-C₆ alkyl, C₁-C₆ alkoxy, C₂-C₆ alkenyl, C₂-C₆ alkynyl, C₂-C₆ alkanoyl, C₂-C₆ alkoxycarbonyl, mono- or di-C₁-C₆ alkylcarboxamide, mono- or di-C₁-C₆ alkylamino, aryl, (aryl)C₁-C₆ alkyl, substituted or unsubstituted (aryl)carbonyl, C₃-C₈ cycloalkyl, halogen, C₁-C₆ haloalkyl, C₁-C₆ haloalkoxy, heteroaryl, (heteroaryl)C₁-C₆ alkyl, heterocycloalkyl, —COOH, —CN, —NH₂, —NO₂, (C₁-C₆ alkyl)amido, (C₂-C₆ alkenyl)amido, (C₂-C₆ alkynyl)amido, or R³ and R⁴ together can form a substituted or unsubstituted C₃-C₆ cycloalkyl, or substituted or unsubstituted C₃-C₆ heterocycloalkyl;
each instance of R⁵ is hydrogen, —OH, C₁-C₆ alkyl, C₁-C₆ alkoxy, C₂-C₆ alkenyl, C₂-C₆ alkynyl, C₂-C₆ alkanoyl, C₂-C₆ alkoxycarbonyl, mono- or di-C₁-C₆ alkylcarboxamide, mono- or di-C₁-C₆ alkylamino, aryl, (aryl)C₁-C₆ alkyl, substituted or unsubstituted (aryl)carbonyl, C₃-C₈ cycloalkyl, halogen, C₁-C₆ haloalkyl, C₁-C₆ haloalkoxy, heteroaryl, (heteroaryl)C₁-C₆ alkyl, heterocycloalkyl, —COOH, —CN, —NH₂, —NO₂, (C₁-C₆ alkyl)amido, (C-C₆ alkenyl)amido, or (C₂-C₆ alkynyl)amido; n is 0, 1, 2, or 3; or a pharmaceutically acceptable salt thereof; with the proviso that when R² and R⁵ are both hydrogen then R¹, R³, and R⁴ are not —OH, methyl, and methyl, respectively.

20. The composition of claim 19, wherein the inhibitor of HMGB2 is a micro RNA or an interfering RNA that inhibits the expression of HMGB2 and the anionic liposome particle is coated with a positively-charged species.

21. The composition of claim 20, wherein the micro RNA is miR-127, miR-539, miR-543, or miR-23a.

22. The composition of claim 20, wherein the interfering RNA is a small hairpin RNA.
